(19) United States
(12) Patent Application Publication
(10) Pub. No.: US 2025/0381207 A1
(43) Pub. Date: Dec. 18, 2025

(54) FLAVONOID-CONTAINING COMPOSITIONS AND TREATMENT OF VIRAL DISEASES WITH SAME

(71) Applicant: AMH Biotech LLC, El Dorado Hills, CA (US)

(72) Inventor: Ali Mohseni Motlagh, Newtown Square, PA (US)

(73) Assignee: AMH Biotech LLC, El Dorado Hills, CA (US)

(21) Appl. No.: 19/237,691

(22) Filed: Jun. 13, 2025

Related U.S. Application Data
(60) Provisional application No. 63/659,539, filed on Jun. 13, 2024.

(51) Int. Cl. A61K 31/7048; A61K 31/198; A61K 31/202; A61K 31/352; A61K 31/375; A61K 31/4525; A61K 31/593; A61K 33/30; A61P 31/14

(52) U.S. Cl. CPC A61K 31/7048; A61K 31/198; A61K 31/202; A61K 31/352; A61K 31/375; A61K 31/4525; A61K 31/593; A61K 33/30; A61P 31/14

(57) ABSTRACT

Compositions that contain flavonoids such as hesperidin, quercetin, hesperetin, and rutin and methods of treating viral diseases such as coronavirus (e.g., SARS COV-2) infection, influenza virus infection, rhinovirus infection, and human metapneumovirus infection.

### CROSS-REFERENCE TO RELATED APPLICATIONS

Priority is hereby claimed to U.S. Provisional Application 63/659,539, filed Jun. 13, 2024, which is incorporated herein by reference in its entirety.

### FIELD OF THE INVENTION

The invention is directed to compositions that contain flavonoids such as hesperidin, quercetin, hesperetin, and rutin and methods of treating viral diseases such as coronavirus (e.g., SARS COV-2) infection, virus infection, rhinovirus infection, and human metapneumovirus infection.

### BACKGROUND

Compositions and methods for treating viral diseases such as coronavirus (e.g., SARS CoV-2) infection, virus infection, rhinovirus infection, and human metapneumovirus with efficacious combinations of flavonoids are needed.

### SUMMARY OF THE INVENTION

One aspect of the invention is directed to compositions. In some versions, the compositions comprise active agents and a carrier, wherein the active agents comprise a first set of flavonoids, wherein the first set of flavonoids consist of: hesperidin; quercetin; and hesperetin, rutin, or a combination of hesperetin and rutin.

In some versions, the composition does not contain at least one of naringin, naringenin, diosmin, quercetin, nobiletin, and tangeretin in an amount more than 1% w/w of the combined mass of the first set of flavonoids.

In some versions, the composition does not contain any one of naringin, naringenin, diosmin, quercetin, nobiletin, and tangeretin in an amount more than 1% w/w of the combined mass of the first set of flavonoids.

In some versions, the composition does not contain any flavonoid other than the flavonoids in the first set of flavonoids in an amount more than 1% w/w of the combined mass of the first set of flavonoids.

In some versions, the first set of flavonoids consists of: hesperidin; quercetin; and rutin or a combination of rutin and hesperetin, and wherein the hesperidin and the rutin are provided in the composition in amounts effective to synergize binding of at least one of the hesperidin and the rutin to SARS-COV-2 spike protein.

In some versions, the first set of flavonoids consists of hesperidin, quercetin, and rutin.

In some versions, the first set of flavonoids consists of hesperidin, quercetin, and hesperetin.

In some versions, the active ingredients further comprise one or more of piperine, a zinc salt, vitamin C, vitamin D3, N-acetylcysteine, and an omega-3 fatty acid.

In some versions, the active ingredients comprise: hesperidin; quercetin; hesperetin, rutin, or a combination of hesperetin and rutin; piperine; a zinc salt; vitamin C; vitamin D3; N-acetylcysteine; and, optionally, one or more omega-3 fatty acids. In some versions, the active ingredients comprise: hesperidin; quercetin; hesperetin; piperine; a zinc salt; vitamin C; vitamin D3; and N-acetylcysteine. In some versions, the active ingredients comprise: hesperidin; quercetin; rutin; piperine; a zinc salt; vitamin C; vitamin D3; N-acetylcysteine; and one or more omega-3 fatty acids.

In some versions, the active ingredients consist of: hesperidin; quercetin; hesperetin, rutin, or a combination of hesperetin and rutin; piperine; a zinc salt; vitamin C; vitamin D3; N-acetylcysteine; and, optionally, one or more omega-3 fatty acids. In some versions, the active ingredients consist of: hesperidin; quercetin; hesperetin; piperine; a zinc salt; vitamin C; vitamin D3; and N-acetylcysteine. In some versions, the active ingredients consist of: hesperidin; quercetin; rutin; piperine; a zinc salt; vitamin C; vitamin D3; N-acetylcysteine; and one or more omega-3 fatty acids.

In some versions, the active ingredients comprise: hesperidin in an amount from 150 mg to 600 mg; quercetin in an amount from 150 mg to 700 mg; hesperetin in an amount from 1 mg to 120 mg, rutin in an amount from 150 mg to 600 mg, or a combination of hesperetin in an amount from 1 mg to 120 mg and rutin in an amount from 150 mg to 600 mg; piperine in an amount from 1 mg to 50 mg; a zinc salt in an amount from 1 mg to 25 mg; vitamin C in an amount from 1 mg to 50 mg; vitamin D3 in an amount from 60 IU to 650 IU; N-acetylcysteine in an amount from 100 mg to 500 mg; and optionally, one or more omega-3 fatty acids in a total amount from 150 mg to 600 mg.

In some versions, the active ingredients consist of: hesperidin in an amount from 150 mg to 600 mg; quercetin in an amount from 150-700 mg; hesperetin in an amount from 1 mg to 120 mg, rutin in an amount from 150 mg to 600 mg, or a combination of hesperetin in an amount from 1 mg to 120 mg and rutin in an amount from 150 mg to 600 mg; piperine in an amount from 1 mg to 50 mg; a zinc salt in an amount from 1 mg to 25 mg; vitamin C in an amount from 1 mg to 50 mg; vitamin D3 in an amount from 60 IU to 650 IU; N-acetylcysteine in an amount from 100 mg to 500 mg; and optionally, one or more omega-3 fatty acids in a total amount from 150 mg to 600 mg.

In some versions, the composition is in a form of a capsule or a gel cap.

Another aspect of the invention is directed to methods of treating a viral infection in a subject. The methods can comprise administering a composition of the invention to the subject in an amount effective to treat the viral infection. In some versions, the infection is selected from the group consisting of a coronavirus infection, an influenza virus infection, a rhinovirus infection, and a human metapneumovirus infection.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1C. Exemplary flavonoids.

FIG. 2. In silico binding of SARS-COV-2 spike protein/human ACE2 complex with hesperidin.

FIG. 3. In silico binding of SARS-COV-2 spike protein/human ACE2 complex with hesperetin.

FIG. 4. In silico binding of SARS-COV-2 spike protein/human ACE2 complex with rutin.

FIG. 5. In silico binding of a complex of SARS-COV-2 TMPRSS2 with hesperidin.

FIG. 6. In silico binding of a complex of SARS-COV-2 TMPRSS2 with rutin.

FIG. 7. In silico binding of a complex of influenza virus haemagglutinin with sialic acid.

FIG. 8. In silico binding of a complex of influenza virus haemagglutinin with hesperidin.

FIG. 9. In silico binding of a complex of influenza virus haemagglutinin with rutin.

FIG. 10. In silico binding of a rhinovirus VP1 antigen/LDLR complex with neohesperidin.

FIG. 11. In silico binding of rhinovirus VP1 antigen (PDB 1R1A) with neohesperidin.

FIG. 12. In silico binding of ICAM1 with neohesperidin.

FIGS. 13A and 13B. SPR binding data with SARS-COV-2 spike protein and hesperidin.

FIG. 13A. SPR trace over time. Rₘₐₓ (RU): 40.4 (calculated), 72.7 (actual). Activity: 40% (calculated), 72% (actual). FIG. 13B. Dose response. Chip Surface: SA. Buffer: TBS-P+ (pH 7.4), 0.5 M ZnCl₂, 2% DMSO. Flow cell temperature: 25° C. Analyte titration: 0.4-100 μM (9-pt; SCK). Flow-rate: 30 μL/min. Target/ligand: SARS-COV2 RBD; 4815 RU captured. Response (theoretical Rₘₐₓ): 101 RU.

FIGS. 14A and 14B. SPR binding data with SARS-COV-2 spike protein and rutin. FIG. 14A. SPR trace over time. Rₘₐₓ (RU): 16.7 (calculated), 21.0 (actual). Activity: 17% (calculated), 21% (actual). FIG. 14B. Dose response. Chip Surface: SA. Buffer: TBS-P+ (pH 7.4), 0.5 μM ZnCl₂, 2% DMSO. Flow cell temperature: 25° C. Analyte titration: 0.4-100 μM (9-pt; SCK). Flow-rate: 30 μL/min. Target/ligand: SARS-COV2 RBD; 4721 RU captured. Response (theoretical Rₘₐₓ): 99.4 RU.

FIGS. 15A and 15B. SPR binding data with SARS-COV-2 spike protein and hesperidin. FIG. 15A. SPR trace over time. Rₘₐₓ (RU): 94.3 (50 μM). Activity: 98% (50 μM). FIG. 15B. Dose response. Chip Surface: SA. Buffer: TBS-P+ (pH 7.4), 0.5 M ZnCl₂, 2% DMSO. Flow cell temperature: 25° C. Analyte titration: 0.4-100 μM (9-pt; SCK). Flow-rate: 30 μL/min. Target/ligand: SARS-COV2 RBD; 4588 RU captured. Response (theoretical Rₘₐₓ): 96.6 RU.

FIG. 16. Normalized viability of Calu-3 cell line pre-treated with inhibitors (100 μM) for 24 hours and incubated with SARS-COV-2 spike protein for 48 hours. Data is normalized to 500 μg/mL puromycin and vehicle (DMEM+1.5%) and represented as a mean and S.E.M. (N=5). Reading is obtained from SpectraMax i3.

FIGS. 17A-17E. Viability of Calu-3 cell line treated with serially diluted inhibitors for 48 hours. FIG. 17A. Normalized viability. Data is normalized to 500 μg/mL puromycin and vehicle (DMEM+1.5%) and represented as a mean and S.E.M. (N=5). Reading is obtained from SpectraMax i3. FIG. 17B. Representative confluency image for Calu-3 (48 hours post dosing) at 100 μM hesperidin. FIG. 17C. Representative confluency image for Calu-3 (48 hours post dosing) at 100 μM rutin. FIG. 17D. Representative confluency image for Calu-3 (48 hours post dosing) at 1:1 hesperidin and rutin. FIG. 17E. Representative confluency image for Calu-3 (48 hours post dosing) at 500 μg/mL puromycin. Inhibitors do not appear to be toxic to Calu-3 cells at tested concentrations.

FIG. 18. In silico binding of SARS-COV-2 spike protein with epigallocatechin gallate (EGCG).

FIG. 19. In silico binding of SARS-COV-2 spike protein with apigenin.

FIG. 20. In silico binding of SARS-COV-2 spike protein with hesperetin.

FIG. 21. In silico binding of SARS-COV-2 spike protein with quercitrin.

FIG. 22. In silico binding of SARS-COV-2 spike protein with surfen.

FIG. 23. In silico binding of SARS-COV-2 spike protein with quillaja.

FIG. 24. SARS-COV-2 spike protein (left panel) and in silico analysis of SARS-COV-2 spike protein and narirutin (right panel), showing no binding.

FIG. 25. In silico binding of human metapneumovirus F protein (prefusion) with luteolin.

FIGS. 26A and 26B. In silico binding of human metapneumovirus F protein (prefusion) with narirutin.

FIG. 27. In silico binding of human metapneumovirus F protein (prefusion) with quercitrin.

FIG. 28. In silico binding of human metapneumovirus F protein (prefusion) with hesperidin.

FIG. 29. In silico binding of human metapneumovirus F protein (prefusion) with quercetin.

FIG. 30. In silico binding of human metapneumovirus G protein with apigenin.

FIG. 31. In silico binding of human metapneumovirus G protein with quercetin.

FIG. 32. In silico binding of human metapneumovirus G protein with quercitrin.

FIG. 33. In silico binding of human metapneumovirus G protein with hesperidin.

### DETAILED DESCRIPTION OF THE INVENTION

On aspect of the invention is directed to compositions comprising active agents. The active agents of the invention preferably comprise flavonoids. The flavonoids preferably comprise various combinations of hesperidin, quercetin, hesperetin, and rutin. The structure of hesperidin is:

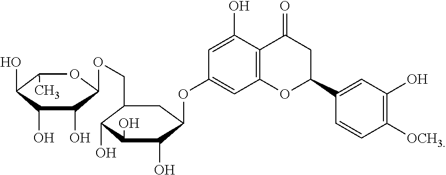

The structure of quercetin is:

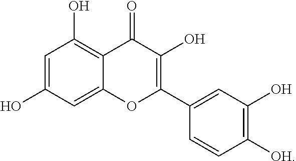 [SMILES: O=c1c(O)c(-c2ccc(O)c(O)c2)oc2cc(O)cc(O)c12]

The structure of hesperetin is:

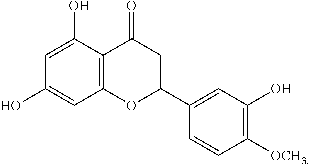 [SMILES: COc1ccc(C2CC(=O)c3c(O)cc(O)cc3O2)cc1O]

The structure of rutin is:

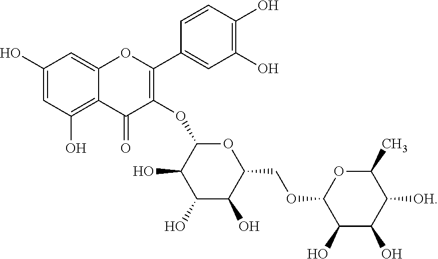 [SMILES: C[C@@H]1O[C@@H](OC[C@H]2O[C@@H](Oc3c(-c4ccc(O)c(O)c4)oc4cc(O)cc(O)c4c3=O)[C@H](O)[C@@H](O)[C@@H]2O)[C@H](O)[C@H](O)[C@H]1O]

The hesperidin, quercetin, hesperetin, and/or rutin can be considered to form a first set of flavonoids that consist of: hesperidin; quercetin; and hesperetin, rutin, or a combination of hesperetin and rutin. In some versions, the first set of flavonoids consists of hesperidin; quercetin; hesperetin, rutin, or a combination of hesperetin and rutin; and neohesperidin. The structure of neohesperidin is:

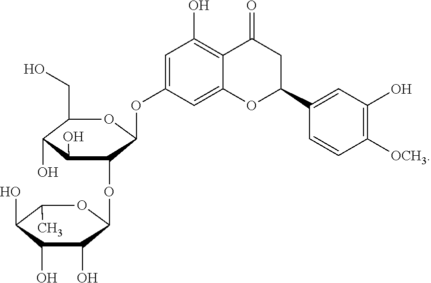

In some versions, the compositions of the invention include hesperidin in an amount from 25 mg to 5,000 mg, such as from 85 mg to 1,500 mg, about 250 mg, or about 250 mg to about 500 mg. In some versions, the compositions of the invention include hesperidin in an amount from 150 mg to about 600 mg.

In some versions, the compositions of the invention include quercetin in an amount from 25 mg to 5,000 mg, such as from 85 mg to 1,500 mg, about 250 mg, or about 400 mg to about 600 mg. In some versions, the compositions of the invention include quercetin in an amount from 150 mg to about 700 mg.

In some versions, the compositions of the invention include hesperidin in an amount from 6 mg to 650 mg, such as from 20 mg to 200 mg, or about 65 mg. In some versions, the compositions of the invention include hesperidin in an amount from 1 about mg to about 120 mg.

In some versions, the compositions of the invention include rutin in an amount from 25 mg to 5,000 mg, such as from 85 mg to 1,500 mg, about 250 mg, or about 250 mg to about 500 mg. In some versions, the compositions of the invention include rutin in an amount from 150 mg to about 600 mg.

The flavonoids in the first set of flavonoids are preferably included in the composition in a purified form such that one or more flavonoids that may normally present with them are not included in the composition.

In various versions of the invention, for example, the composition does not contain at least one of naringin, naringenin, diosmin, nobiletin, and tangeretin in an amount more than 25% w/w, 20%, w/w, 15%, w/w, 10% w/w, 5% w/w, 1% w/w, 0.5% w/w, 0.1% w/w, 0.05% w/w, 0.01% w/w, 0.005% w/w, or 0.001% w/w of the combined mass of the first set of flavonoids. In some versions of the invention, the composition is completely devoid of at least one of naringin, naringenin, diosmin, nobiletin, and tangeretin.

In various versions of the invention, the composition does not contain any one of naringin, naringenin, diosmin, nobiletin, and tangeretin in an amount more than 25% w/w, 20%, w/w, 15%, w/w, 10% w/w, 5% w/w, 1% w/w, 0.5% w/w, 0.1% w/w, 0.05% w/w, 0.01% w/w, 0.005% w/w, or 0.001% w/w of the combined mass of the first set of flavonoids. In some versions, of the invention, the composition is completely devoid of each of naringin, naringenin, diosmin, nobiletin, and tangeretin.

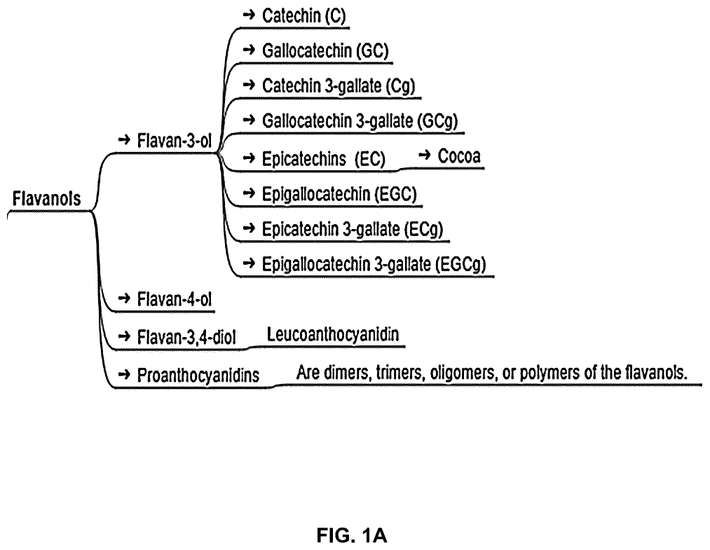

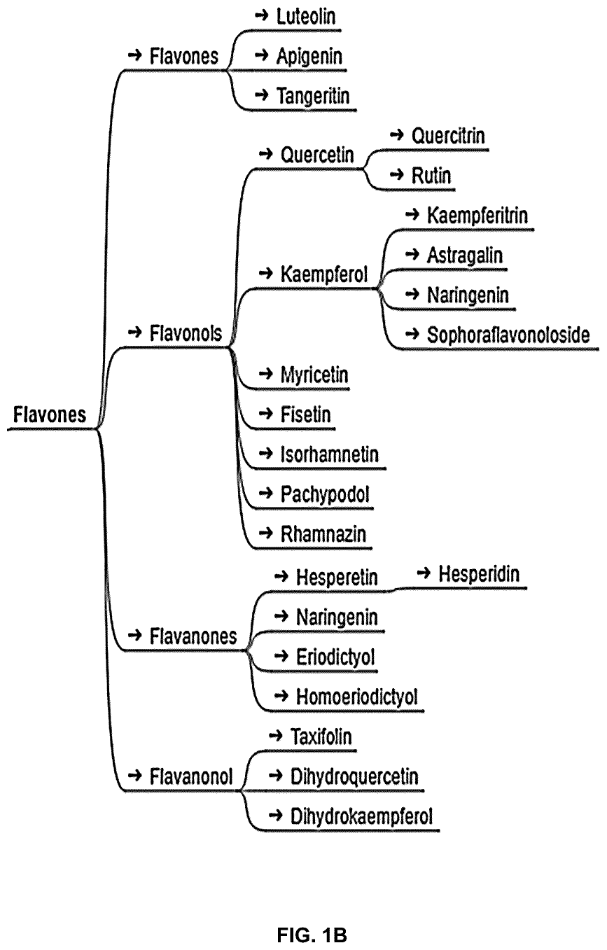

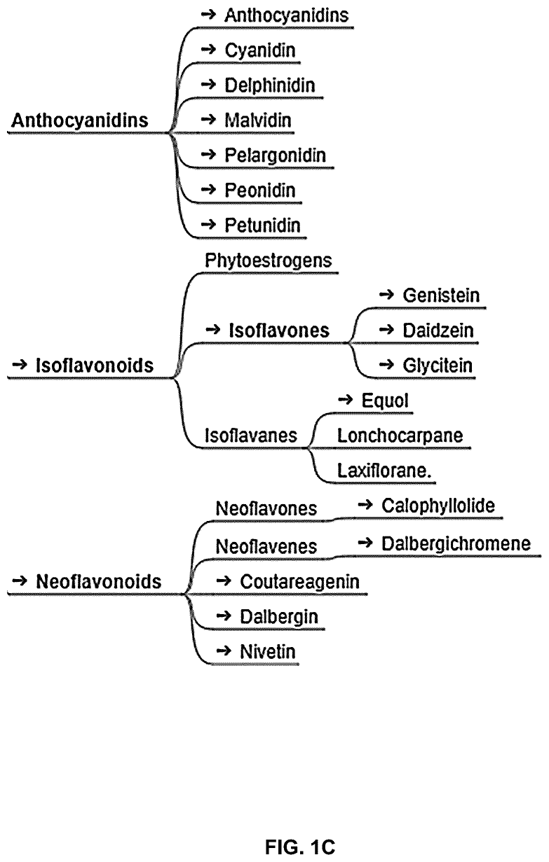

In various versions of the invention, the composition does not contain any one or more flavonoids shown in FIGS. 1A-1C other than the flavonoids of the first set of flavonoids in an amount more than 25% w/w, 20%, w/w, 15%, w/w, 10% w/w, 5% w/w, 1% w/w, 0.5% w/w, 0.1% w/w, 0.05% w/w, 0.01% w/w, 0.005% w/w, or 0.001% w/w of the combined mass of the first set of flavonoids. In some versions of the invention, the composition is completely devoid of any one or more flavonoids shown in FIGS. 1A-1C other than the flavonoids of the first set of flavonoids.

In various versions of the invention, the composition does not contain any flavonoids shown in FIGS. 1A-1C other than the flavonoids of the first set of flavonoids in an amount more than 25% w/w, 20%, w/w, 15%, w/w, 10% w/w, 5% w/w, 1% w/w, 0.5% w/w, 0.1% w/w, 0.05% w/w, 0.01% w/w, 0.005% w/w, or 0.001% w/w of the combined mass of the first set of flavonoids. In some versions of the invention, the composition is completely devoid of each of the flavonoids shown in FIGS. 1A-1C other than the flavonoids of the first set of flavonoids.

In various versions of the invention, the composition does not contain any flavonoid other than the flavonoids of the first set of flavonoids in an amount more than 25% w/w, 20%, w/w, 15%, w/w, 10% w/w, 5% w/w, 1% w/w, 0.5% w/w, 0.1% w/w, 0.05% w/w, 0.01% w/w, 0.005% w/w, or 0.001% w/w of the combined mass of the first set of flavonoids. In some versions of the invention, the composition is completely devoid of any flavonoid other than the flavonoids of the first set of flavonoids.

As used herein, the term “flavonoid” refers to compounds have the general structure of a 15-carbon skeleton, which consists of two phenyl rings (A and B) and a heterocyclic ring (C, the ring containing the embedded oxygen). This carbon structure can be abbreviated C6-C3-C6. According to the IUPAC nomenclature. An exemplary flavonoid carbon skeleton is as follows:

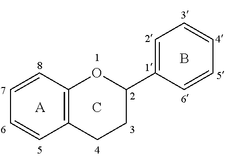

Exemplary flavonoids and types of flavonoids are provided in FIGS. 1A-1C.

As shown in the following examples hesperidin and rutin can synergize their binding to certain proteins, such as the SARS-COV-2 spike protein. Accordingly, some versions of the invention include the hesperidin and the rutin in the composition in amounts effective to synergize binding of at least one of the hesperidin and the rutin to SARS-COV-2 spike protein.

In some versions, the compositions of the invention can comprise any one or more flavonoids shown or predicted (e.g., via in silico analysis) in the following examples as binding any one or more of the SARS-COV-2 spike protein, SARS-COV-2 TMPRSS2, influenza virus haemagglutinin, rhinovirus VP1 antigen, ICAM1, human metapneumovirus F protein, human metapneumovirus G protein, and human metapneumovirus IFG-1R.

The compositions of the invention can comprise other active agents other than flavonoids. Such active agents can include, for example, any one or more of piperine, zinc, vitamin C, vitamin D3, N-acetylcysteine, and one or more omega-3 fatty acids.

In some versions, the compositions of the invention comprise piperine. The piperine can be included in the compositions in the form of BIOPERINE® (Sabinsa Corporation, East Windsor, NJ). In some versions, the piperine is included in the composition in an amount from 0.5 mg to 100 mg, such as from 2 mg to 30 mg, about 5 mg, or from about 5 mg to about 10 mg. In some versions, the piperine is included in the composition in an amount from 1 mg to 50 mg.

In some versions, the composition comprises zinc. The zinc can be in the form of a zinc salt, such as zinc citrate. In some versions, the zinc salt is included in the composition in an amount from 1.5 mg to 150 mg, such as from 5 mg to 50 mg or about 15 mg. In some versions, the zinc salt is included in the composition in an amount from 1 mg to 50 mg or from 1 mg to 25 mg.

In some versions, the composition comprises vitamin C (ascorbic acid). In some versions, the vitamin C is included in the composition in an amount from 3 mg to 300 mg, such as from 10 mg to 100 mg or about 30 mg. In some versions, the vitamin C is included in the composition in an amount from 1 mg to 50 mg.

In some versions, the composition comprises vitamin D3 (cholecalciferol). In some versions, the vitamin D3 is included in the composition in an amount from 20 International Units (IU) μg to 2,000 IU, such as 60 IU to 650 IU, or about 200 IU.

In some versions, the composition comprises N-acetylcysteine. In some versions, the N-acetylcysteine is included in the composition in an amount from 20 mg to 4,000 mg, such as from 60 mg to 1,200 mg, about 300 mg, or from about 200 mg to about 400 mg. In some versions, the N-acetylcysteine is included in the composition in an amount from 100 mg to 500 mg.

In some versions, the composition comprises one or more omega-3 fatty acids. The omega-3 fatty acids can comprise eicosapentaenoic acid (EPA), docosahexaenoic acid (DHA), or a combination thereof, among others. In some versions, the omega-3 fatty acids are included in the composition in an amount from 25 mg to 5,000 mg, such as from 85 mg to 1,500 mg, about 250 mg, or about 250 mg to about 500 mg. In some versions, the omega-3 fatty acids are included in the composition in an amount from 150 mg to about 600 mg.

Some aspects of the invention are directed to methods of treating a viral infection in a subject. The methods can comprise administering a composition of the invention to the subject in an amount effective to treat the viral infection. In various embodiments, the viral infection can be an infection of a hepatitis virus (hepatitis A virus, hepatitis B virus, hepatitis C virus), coronavirus (e.g., SARS-COV-2), influenza virus, herpes simplex virus, varicella-zoster virus, paramyxovirus, human papillomavirus (HPV), measles virus, respiratory syncytial virus, rubella virus, human immunodeficiency virus, rhinovirus, human metapneumovirus, rotavirus, norovirus, Ebola virus, Junin virus, and/or Zika virus, among others.

In some versions of the invention, the composition is administered to the subject after exposure to the virus of the viral infection. In some versions of the invention, the composition is administered to the subject prior to exposure to the virus of the viral infection. In some versions of the invention, the composition is administered to the subject after onset of the viral infection.

In some versions of the invention, the composition is administered to the subject prior to onset of the viral infection. In some versions of the invention, the composition is administered to the subject after onset of the viral infection. In some versions of the invention, the composition is administered to the subject prior to onset of the viral infection. “Onset” of the viral infection can refer to positive detection of the virus in the subject, appearance of a symptom of the viral infection, or a combination thereof.

The following definitions apply to the compositions, methods, and uses described herein unless the context clearly indicates otherwise, and it is to be understood that the claims may be amended to include language within a definition as needed or desired. Moreover, the definitions apply to linguistic and grammatical variants of the defined terms (e.g., the singular and plural forms of a term), and some linguistic variants are particularly mentioned below (e.g., “administration” and “administering”).

The term “about,” when used in reference to a value, signifies any value or range of values that is plus-or-minus 10% of the stated value (e.g., within plus-or-minus 1%, 2%, 3%, 4%, 5%, 6%, 7%, 8%, 9% or 10% of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). As another example, a prevalence rank in a population of about 80% means a prevalence rank of 72-88% (e.g., 79.2-80.8%). In case of doubt, “about X” can be “X” (e.g., about 80% can be 80%). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure, etc.)). In the event an instrument or technique measuring a value has a margin of error greater than 10%, a given value will be about the same as a stated value when they are both within the margin of error for that instrument or technique.

The term “administration” and variants thereof, such as “administering,” refer to the administration of the flavonoids described herein, or a composition containing the flavonoids to a subject (e.g., a human patient). As a result of the administration, the flavonoids or composition containing the flavonoids (e.g., a pharmaceutical composition) are introduced to the subject. In addition to compositions of the invention and additional active agents useful in combination therapies, items used as positive controls, negative controls, and placebos, any of which can also be a compound, can also be “administered.” One of ordinary skill in the art will be aware of a variety of routes that can, in appropriate circumstances, be utilized for administration to a subject. For example, the route of administration can be oral (i.e., by swallowing a composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours).

One of ordinary skill in the art will appreciate that the term “dosage form” may be used to refer to a physically discrete unit of an active agent (e.g., a flavonoid or combination of flavonoids) for administration to a patient. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular patient is determined by one or more attending physicians and may involve administration of multiple dosage forms.

One of ordinary skill in the art will appreciate that the term “dosing regimen” may be used to refer to a set of unit doses (typically more than one) that are administered individually to a patient, separated by equal or unequal periods of time. A given therapeutic agent typically has a recommended dosing regimen, which may involve one or more doses, each of which may contain the same unit dose amount or differing amounts. In some embodiments, a dosing regimen comprises a first dose in a first dose amount, followed by one or more additional doses in a second dose amount that is different from the first dose amount. In some embodiments, a dosing regimen is correlated with a desired or beneficial outcome when administered across a relevant population (i.e., the regimen is a therapeutic dosing regimen).

As used herein, an “effective amount” of an active agent refers to an amount that produces or is expected to produce the desired effect for which it is administered. The effective amount will vary depending on factors such as the desired biological endpoint, the pharmacokinetics of the active agent administered, the condition being treated, the mode of administration, and characteristics of the patient, as discussed further below and recognized in the art. The term can be applied to therapeutic and prophylactic methods. For example, a therapeutically effective amount is one that reduces the incidence and/or severity of one or more signs or symptoms of the disease. One of ordinary skill in the art will appreciate that the term does not in fact require successful treatment be achieved in any particular individual. Rather, a therapeutically effective amount is that amount that provides a particular desired pharmacological response in a significant number of patients when administered to patients in need of such treatment. In some embodiments, reference to a therapeutically effective amount may be a reference to an amount administered or an amount measured in one or more specific tissues (e.g., a tissue affected by the disease) or fluids (e.g., blood, saliva, serum, sweat, tears, urine, etc.). Effective amounts may be formulated and/or administered in a single dose or in a plurality of doses, for example, as part of a dosing regimen.

The term “patient” and “subject” are used interchangeably herein and refer to any organism that is or may be subjected to the administration of an active agent described herein. The administration may be administered for, e.g., experimental, diagnostic, prophylactic, and/or therapeutic purposes. Typical patients include animals (e.g., mammals such as mice, rats, rabbits, non-human primates, and humans; domesticated animals, such as dogs and cats; and livestock or any other animal of agricultural or commercial value). A patient may be suffering from or be susceptible to (i.e., have a higher than average risk of developing) a disease described herein and may display one or more signs or symptoms thereof.

The term “pharmaceutically acceptable,” when applied to a carrier used to formulate a composition disclosed herein (e.g., a pharmaceutical composition), means a carrier that is compatible with the other ingredients of the composition and not deleterious to a patient (e.g., it is non-toxic in the amount required and/or administered (e.g., in a unit dosage form)).

The term “pharmaceutically acceptable,” when applied to a salt, solvate, stereoisomer, tautomer, or isotopic form of an active agent described herein, refers to a salt, solvate, stereoisomer, tautomer, or isotopic form that is, within the scope of sound medical judgment, suitable for use in contact with the tissues of humans (e.g., patients) and lower animals (including, but not limited to, mice and rats used in laboratory studies) without unacceptable toxicity, irritation, allergic response and the like, and that can be used in a manner commensurate with a reasonable benefit/risk ratio. Many pharmaceutically acceptable salts are well known in the art.

Pharmaceutically acceptable salts of the active agent s described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALAT1e, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “reference” used with regard to an agent, patient, population, sample, or value, etc., refers to a standard or control relative to which a comparison is performed. For example, an agent, patient, population, sample, or value of interest is compared with a reference agent, patient, population, sample, or value. The reference can be analyzed or determined substantially simultaneously with the analysis or determination of the item of interest or it may constitute a historical standard or control, determined at an earlier point in time and optionally embodied in a tangible medium. One of ordinary skill in the art is well trained in selecting appropriate references, which are typically determined or characterized under conditions that are comparable to those encountered by the item of interest. One of ordinary skill in the art will appreciate when sufficient similarities are present to justify reliance on and/or comparison to a particular possible reference as a standard or control.

As used herein, a “response” to treatment is any beneficial alteration in a patient's condition that results from, or that correlates with, treatment. The alteration may be stabilization of the condition (e.g., inhibition of deterioration that would have taken place in the absence of the treatment), amelioration of, delay of onset of, and/or reduction in frequency of one or more signs or symptoms of the condition, improvement in the prospects for cure of the condition, greater survival time, etc.

As used herein, the terms “treatment,” “treat,” and “treating” refer to reversing, alleviating, delaying the onset of, and/or inhibiting the progress of a “pathological condition” (e.g., a disease, such as a viral infection) described herein. In some embodiments, “treatment,” “treat,” and “treating” require that signs or symptoms of the disease have developed or have been observed. In other embodiments, treatment may be administered in the absence of signs or symptoms of the disease or condition (e.g., in light of a history of symptoms and/or in light of genetic or other susceptibility factors). Treatment may also be continued after symptoms have resolved, for example, to delay or inhibit recurrence.

Relative amounts of the active agent/ingredient, the pharmaceutically acceptable carrier(s), and/or any additional ingredients in a composition of the invention can vary, depending upon the identity, size, and/or condition of the subject treated and further depending upon the route by which the composition is to be administered and the disease to be treated. By way of example, the composition may comprise between about 0.1% and 99.9% (w/w or w/v) of an active agent/ingredient.

Pharmaceutically acceptable carriers useful in the manufacture of the compositions described herein are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable carriers useful in the manufacture of the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions used as described herein may be administered orally. Such orally acceptable dosage forms may be solid (e.g., a capsule, tablet, sachet, powder, granule, and orally dispersible film) or liquid (e.g., an ampoule, semi-solid, syrup, suspension, or solution (e.g., aqueous suspensions or dispersions and solutions). In the case of tablets, carriers commonly used include lactose and corn starch. Lubricating agents, such as magnesium stearate, can also be included. In the case of capsules, useful diluents include lactose and dried cornstarch. When aqueous suspensions are formulated, the active agent/ingredient can be combined with emulsifying and suspending agents. In any oral formulation, sweetening, flavoring or coloring agents may also be added. In any of the various embodiments described herein, an oral formulation can be formulated for immediate release or sustained/delayed release and may be coated or uncoated. A provided composition can also be micro-encapsulated.

Compositions suitable for buccal or sublingual administration include tablets, lozenges, and pastilles. Formulations can also be prepared for subcutaneous, intravenous, intramuscular, intraocular, intravitreal, intra-articular, intra-synovial, intrasternal, intrathecal, intrahepatic, intraperitoneal intralesional and by intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Although the descriptions of compositions provided herein are principally directed to compositions which are suitable for administration to humans, it will be understood by one of ordinary skill in the art that such compositions are generally suitable for administration to animals of all sorts. Modification of compositions suitable for administration to humans in order to render the compositions suitable for administration to various animals is well understood, and the ordinarily skilled veterinary pharmacologist can design and/or perform such modification.

Active agents described herein are typically formulated in dosage unit form, e.g., single unit dosage form, for ease of administration and uniformity of dosage. The specific therapeutically or prophylactically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The amount of an active agent required to achieve an optimum clinical outcome can vary from subject to subject, depending, for example, on species, age, and general condition of a subject, severity of the side effects, cancer to be treated, identity of the particular active agent(s) to be administered, and mode of administration. The desired dosage can be delivered two or three times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of an active agent for administration one or more times a day (e.g., once) to a 70 kg adult human may comprise about 1-100 mg, about 1-50 mg, about 1-35 mg (e.g., about 1-5, 1-10, 1-15, 1-20, 1-25, or 1-30 mg), about 2-20 mg, about 3-15 mg or about 10-30 mg (e.g., 10-20 or 10-25 mg). Here, and wherever ranges are referenced, the end points are included. The dosages provided in this disclosure can be scaled for patients of differing weights or body surface and may be expressed per m² of the patient's body surface. In certain embodiments, compositions of the invention may be administered once per day. The dosage of an active agent can be about 1-100 mg, about 1-50 mg, about 1-25 mg, about 2-20 mg, about 5-15 mg, about 10-15 mg, or about 13-14 mg. In certain embodiments, a composition of the invention may be administered twice per day. In some embodiments, the dosage of an active agent for each administration is about 0.5 mg to about 50 mg, about 0.5 mg to about 25 mg, about 0.5 mg to about 1 mg, about 1 mg to about 10 mg, about 1 mg to about 5 mg, about 3 mg to about 5 mg, or about 4 mg to about 5 mg.

Unless the content dictates otherwise, references to any flavonoid herein encompasses salts and/or enantiomers of the structures for the flavonoid provided herein, except that the references to the flavonoids in the Examples refer to the exact structures for the flavonoids provided herein.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1

### Summary

We conducted an in silico study using AutoDock Vina (Trott et al. 2010). We discovered that many citrus flavonoids potentially bind viral pathogens and may hinder cellular entry and infection. We then conducted in vitro ligand binding studies (fluorescence inhibition and surface plasmon resonance (SPR) assays) and confirmed the binding of hesperidin, hesperetin, and rutin to the main antigens of SARS-COV-2 (e.g., spike protein and TMPRSS2), influenza virus, and rhinovirus, thereby indicating their ability to bind pathogenic viruses, block cellular entry, and control viral infection. Our research and laboratory testing indicated that rutin synergized the inhibitory effect of hesperidin on the binding to the SARS-COV-2 spike protein and ACE2 (from 82% activity with hesperidin alone to 98% activity with hesperidin and rutin).

### In Silico Study

AutoDock Vina (Trott O, Olson A J. AutoDock Vina: improving the speed and accuracy of docking with a new scoring function, efficient optimization, and multithreading. J Comput Chem. 2010 Jan. 30; 31(2):455-61) was used for an in silico study. The SARS-COV-2 spike protein, angiotensin converting enzyme 2 (ACE2), and transmembrane protease serine 2 (TMPRSS2) proteins were selected from Research Collaboratory for Structural Bioinformatics Protein Data Bank (RCSB PDB; world wide web at rcsb.org). Flavonoids (ligands) were selected from the National Institutes of Health (NIH) PubChem database (world wide web at pubchem.ncbi.nlm.nih.gov). Protein processing started with visualization using the PYMOL software molecular viewer (world wide web at pymol.org). Theoretical drug binding analysis was performed using the PBD 6VWL (SARS-COV-2) and PDB 7FEM (SARS-COV-2) structures, among others, downloaded from RCSB PDB. PBD 6VWL represents a high-fidelity SARS-COV-2 chimeric receptor-binding domain complexed with its receptor human ACE2.

The in silico molecular docking experiments started with protein and ligand preparation. Prior to the assay, molecules (ligands and proteins) were first edited in AutoDockTools, to remove heteroatoms, repair atoms, add kollman charges, and remove unwanted chains from the analysis. The proteins and ligands were then converted to PDBQT format. Grid dimension was generated by AutoDock Vina. Protein and ligand docking experiments were performed with AutoDock Vina using hesperidin, rutin, and hesperetin as ligands and spike protein (PDB 7FEM), TMPRSS2 enzyme (PDB 7meq), human ICAM1 (PDB lic1), and rhinovirus VP1 (PDB 1R1A), among others, as proteins. An energy range of 4 and an exhaustiveness of 8 were applied to configure the assay.

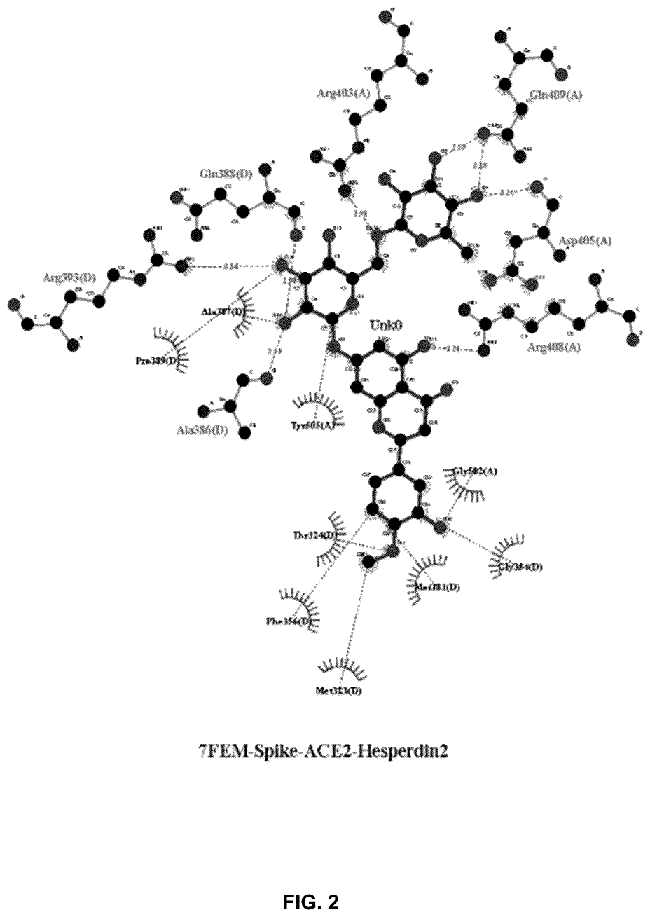

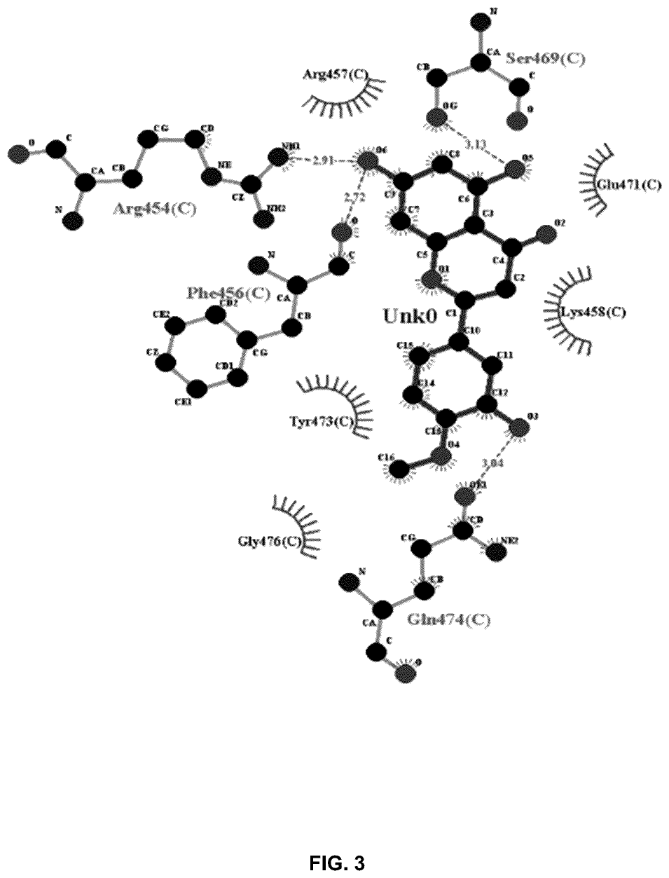

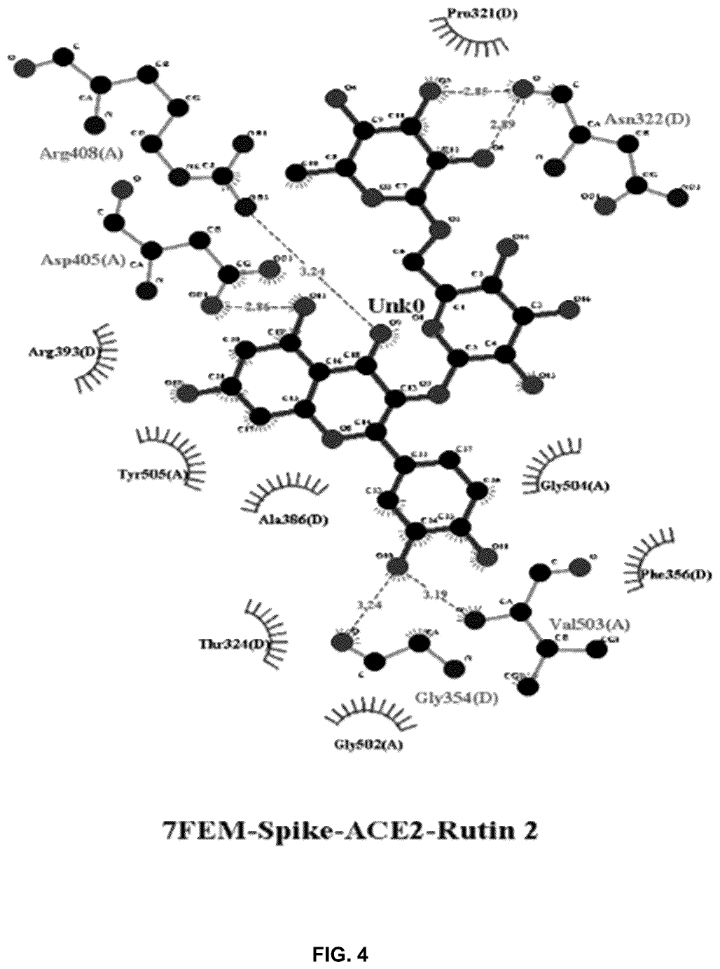

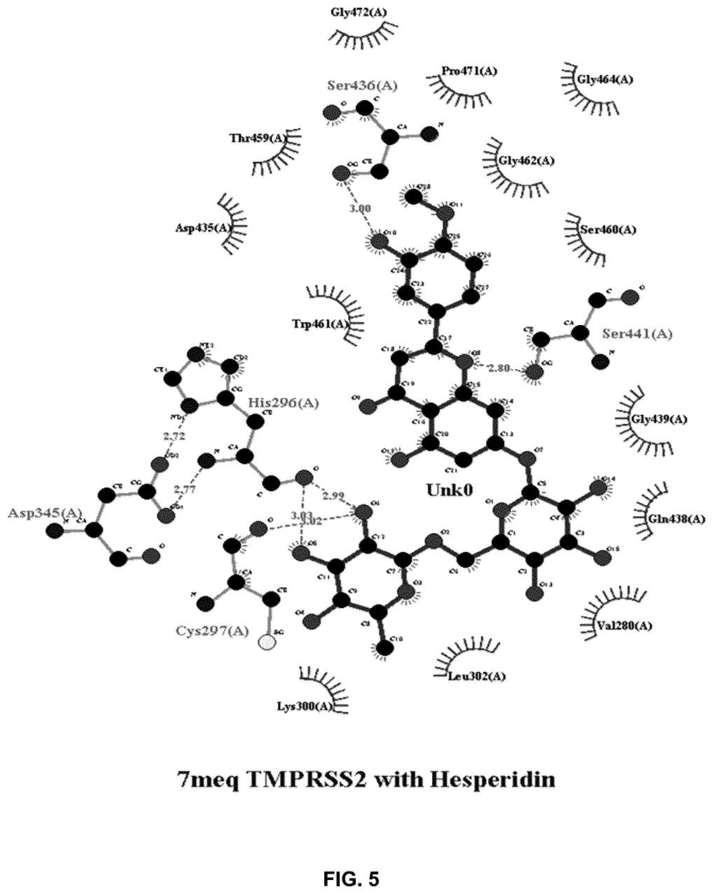

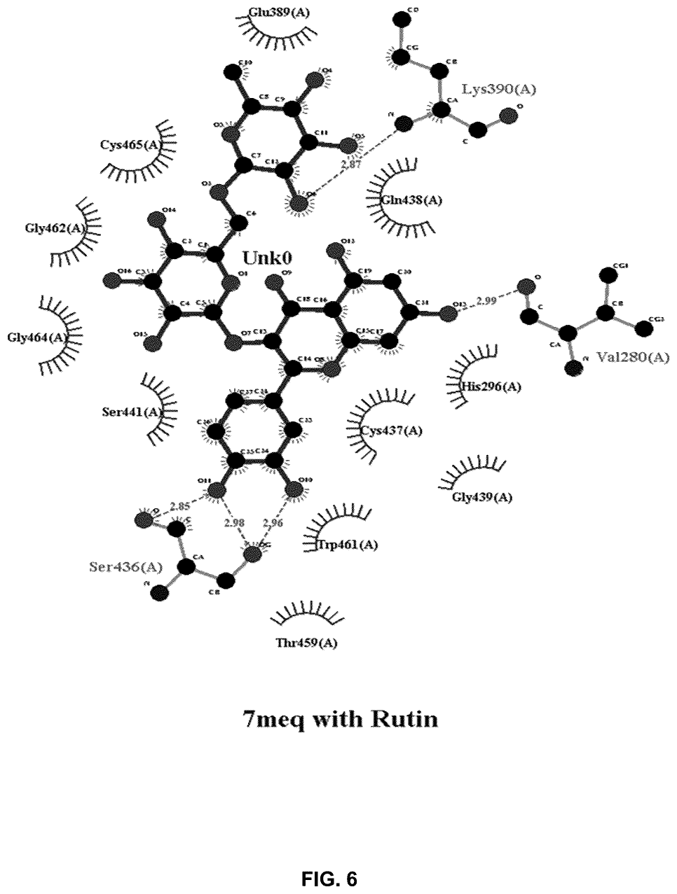

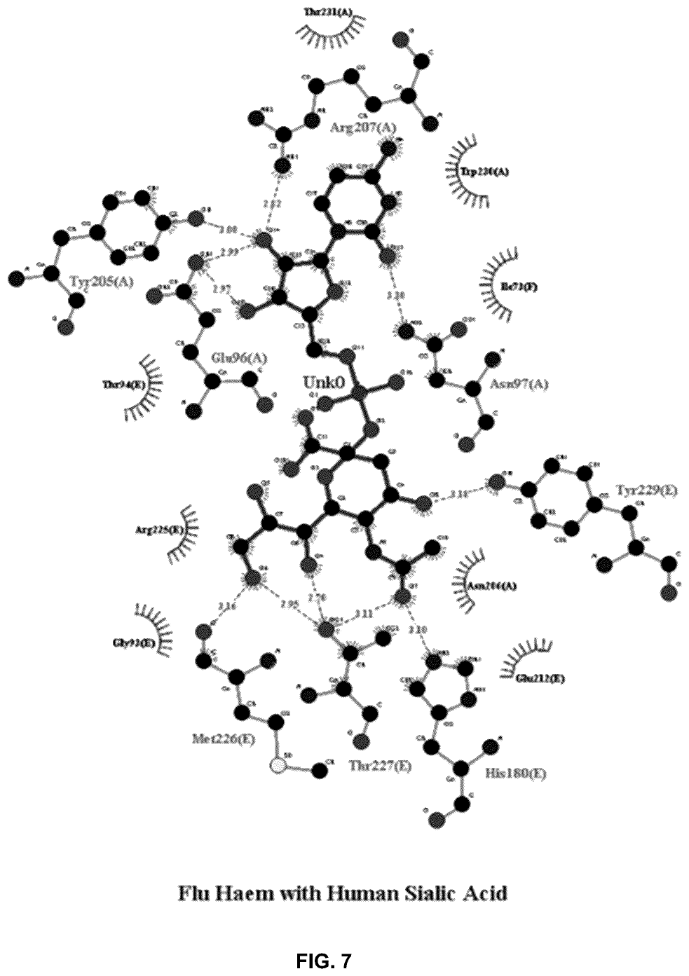

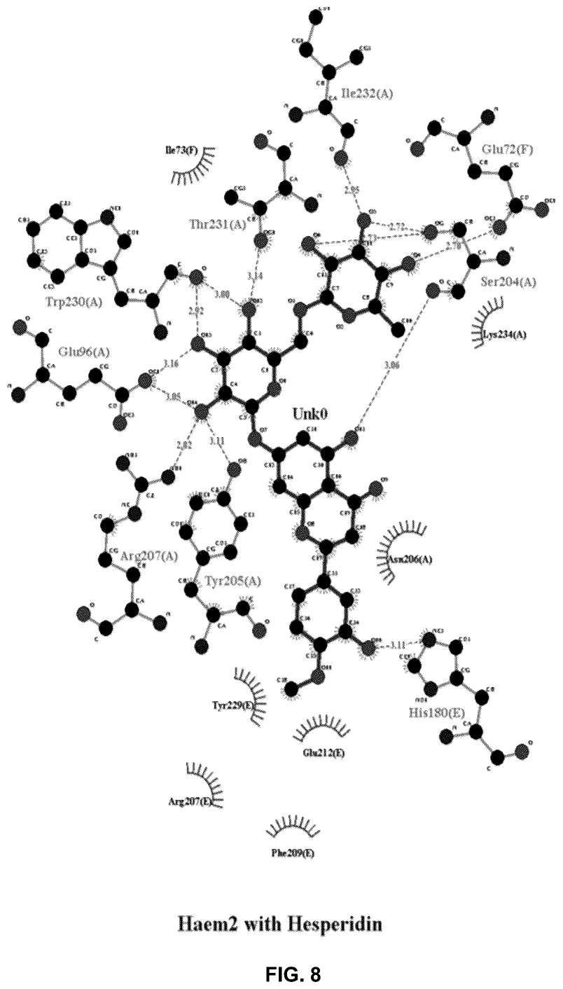

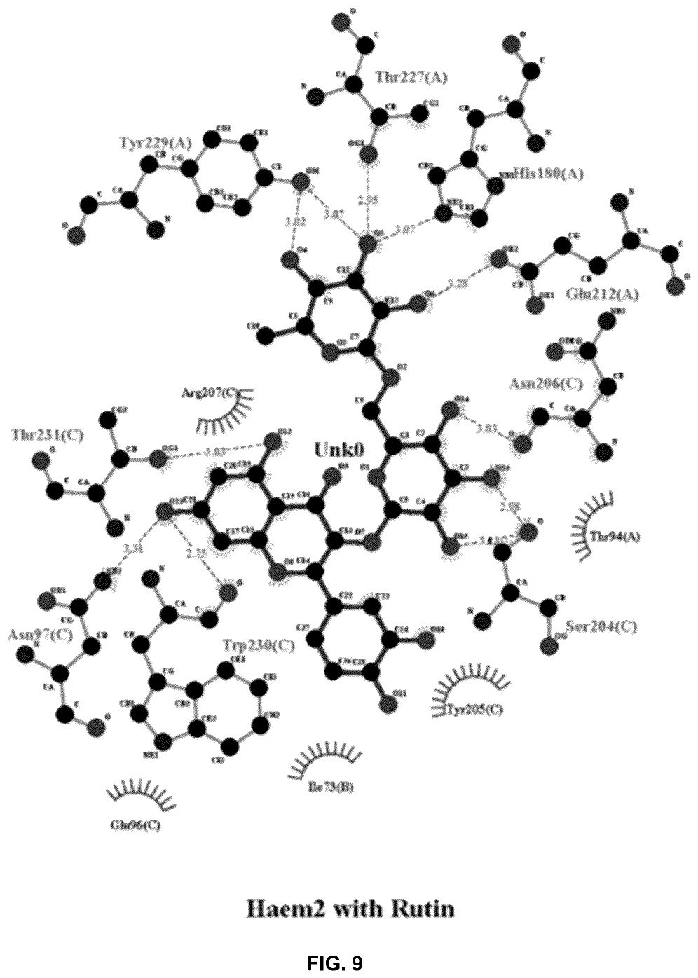

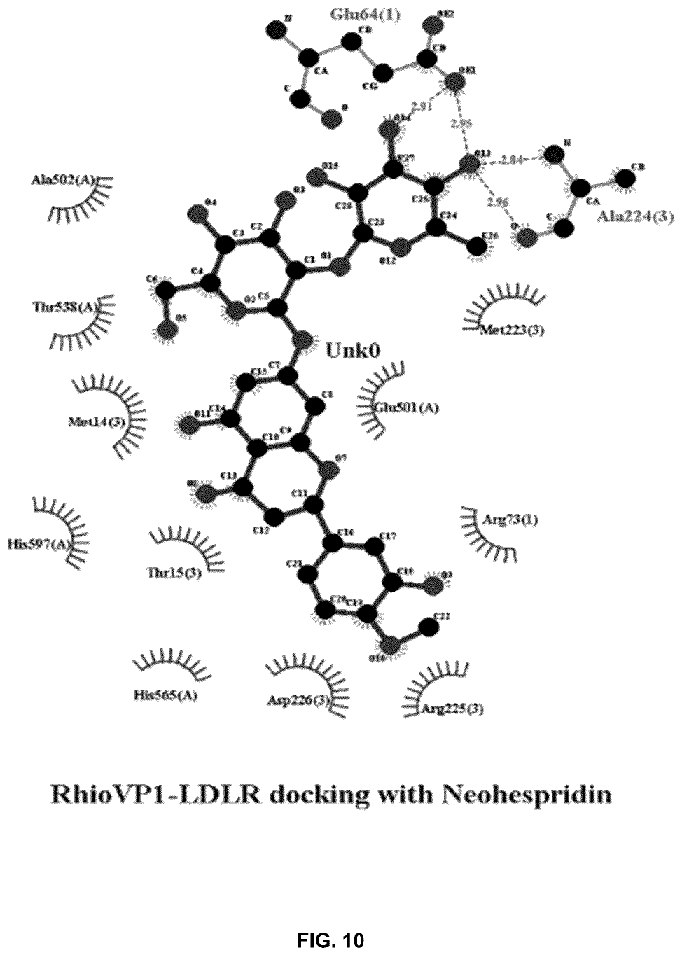

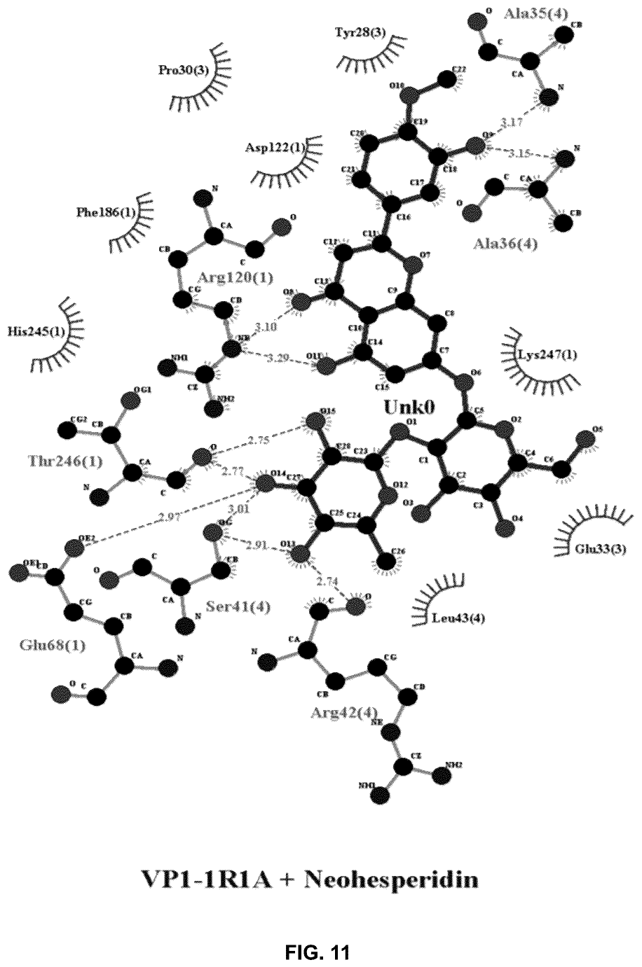

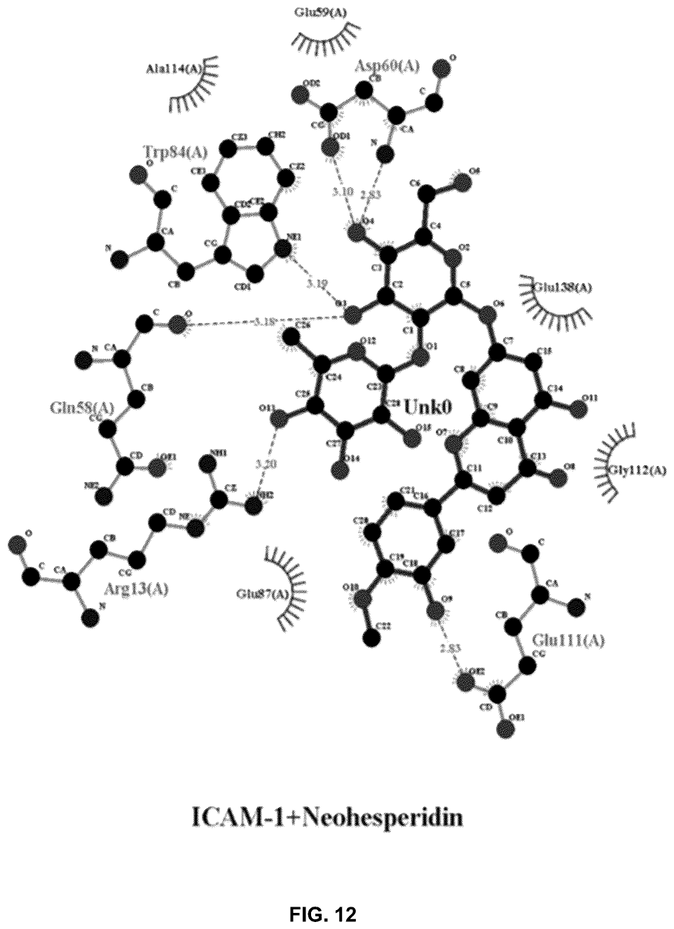

The in silico analysis showed strong association of hesperidin, neohesperidin, hesperetin, and rutin with the spike protein of SARS-COV-2, sialic acid of human epithelial cell, and the rhinovirus VP1 antigen. ACE2 (PDB 7FEM) bound neohesperidin with an affinity of −9.6 Kcal/mol. ACE2 (PDB 7FEM) bound hesperetin with an affinity of −9.5 Kcal/mol. Spike protein of SARS-COV-2 bound with rutin with an affinity −7.6 Kcal/mol. Human LDLR bound neohesperidin with affinity −8.3 Kcal/mol. ICAM1 bound hesperidin with an affinity of −7.0 Kcal/mol. Rhinovirus VP1 (PDB 1R1A) bound neohesperidin with an affinity of −9.1 Kcal/mol. Influenza virus neuraminidase (PDB 2AEQ) bound rutin with an affinity of −9.9 Kcal/mol. Hemagglutinin bound rutin with an affinity of −8.8 Kcal/mol. Hemagglutinin bound neohesperidin with an affinity of −8.5 Kcal/mol. Rhinovirus VP1-LDLR bound neohesperidin with an affinity of −9.0 Kcal/mol. SARS-COV-2 TMPRSS2 enzyme bound hesperidin with an affinity of −7.4 Kcal/mol. Select structure results are shown in FIGS. 2-12.

### In Vitro Fluorescence Binding Assay

A fluorescence ligand bind assay (Lora Benoit, Vinayak Shenoy, Simon Meykler, Ali Mohseni. SARS-COV-2 spike protein 13-mer subdomain corresponds to the drug-binding domain of glutamyl-propyl-tRNA synthetase 1 and is targetable by halofuginone, 22 Jul. 2021, PREPRINT (Version 1) available at Research Square [world wide web at doi.org/10.21203/rs.3.rs-738132/v1]) used to test the binding of various flavonoids. Fluorescence values increased over time (likely due to evaporation at 37° C.), but differences in fluorescence intensity after a kinetic read may be used to calculate relative ACE2 inhibition. Naringenin, nobiletin, and rutin resulted in 57%, 60%, and 44% of control/blank fluorescence at 100 μM and IC₅₀=36.2, 26.5, and 39.4 μM, respectively.

### Surface Plasmon Resonance (SPR) Analysis

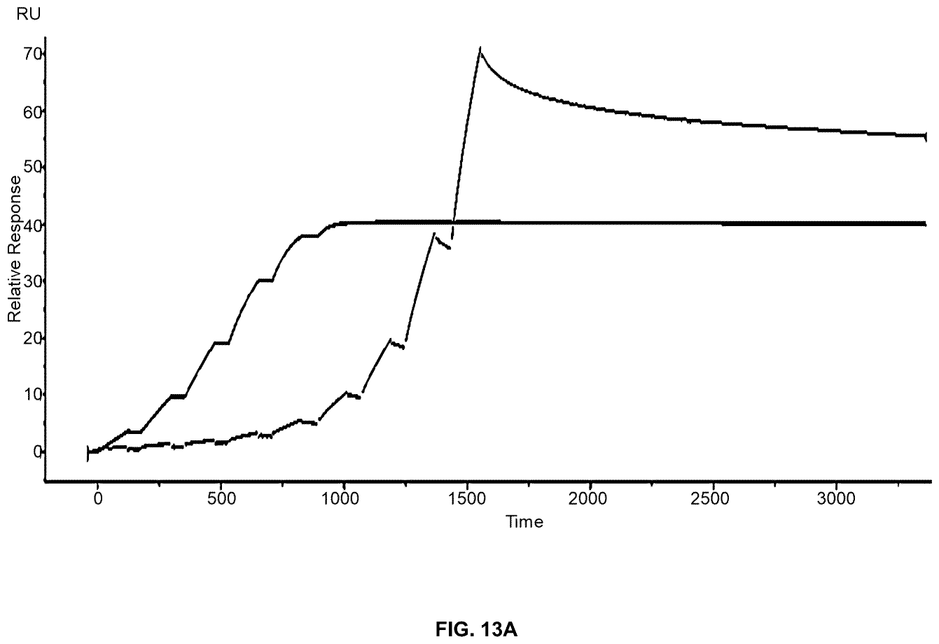

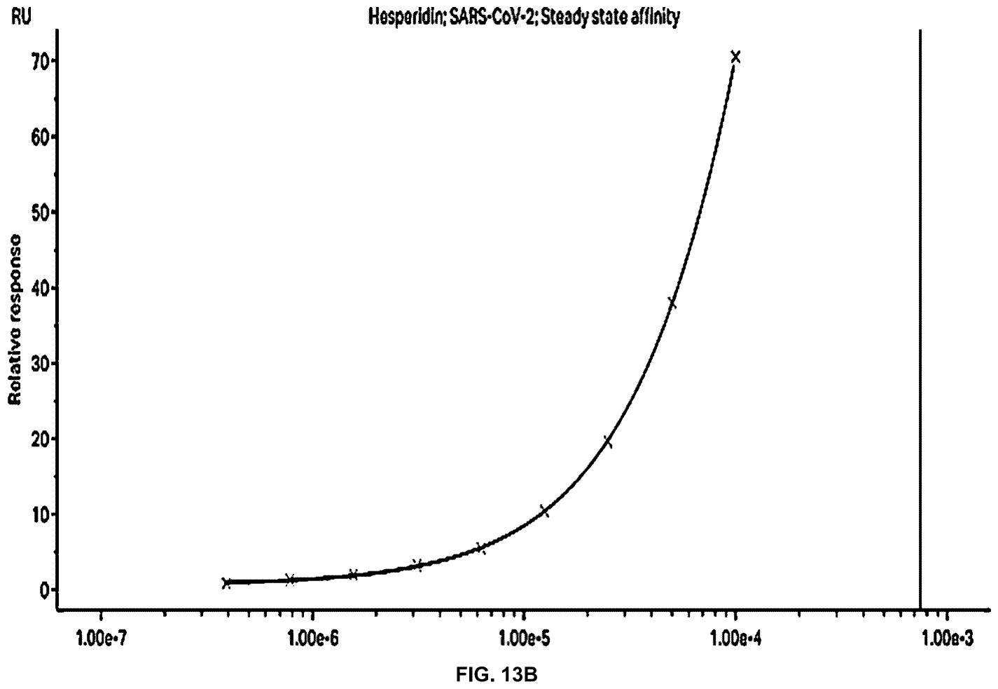

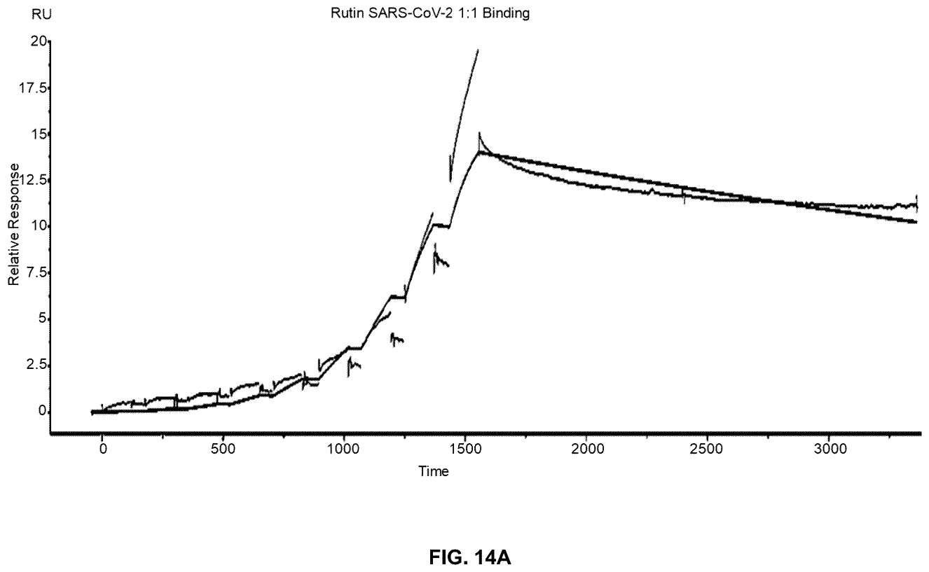

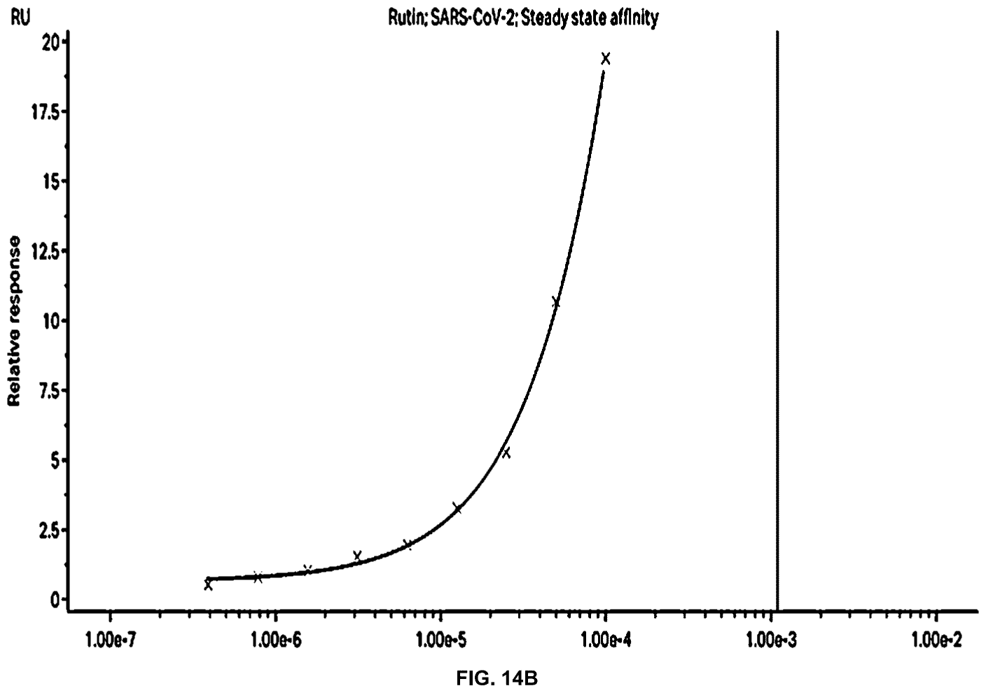

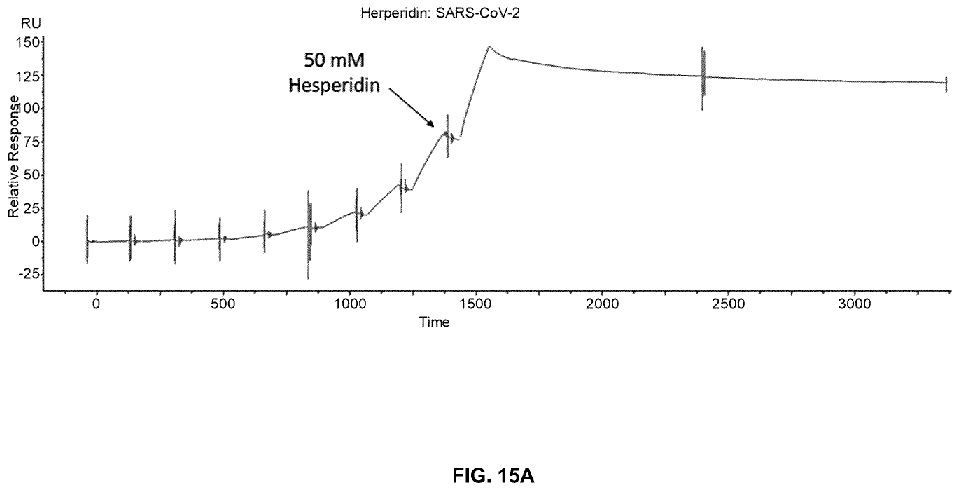

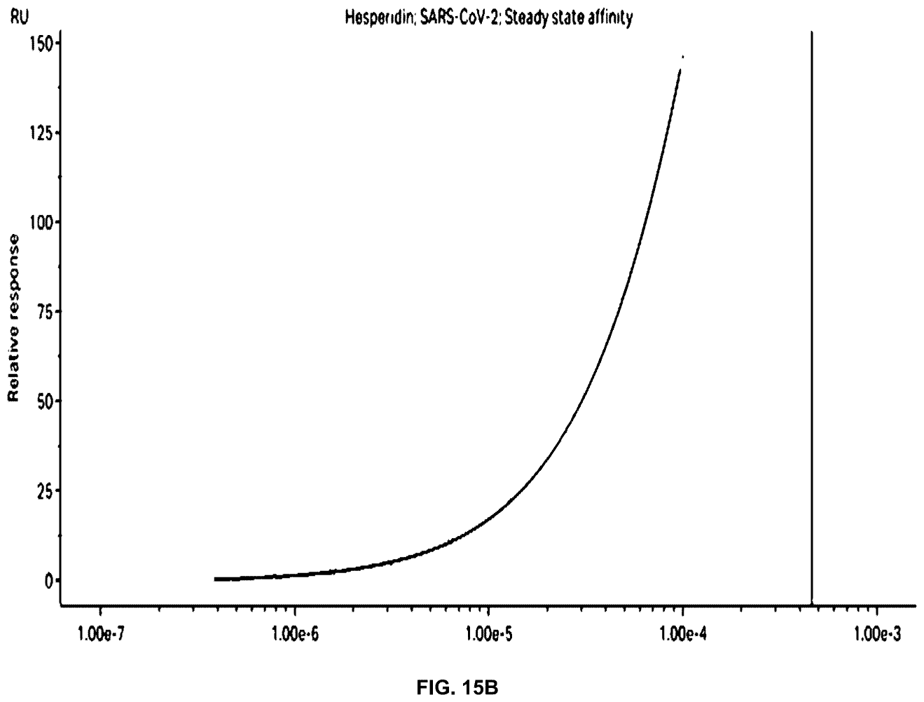

Recombinant SARS-COV-2 spike protein (Catalog No. AVI10500, R&D Systems, Inc., Minneapolis, MN) and ACE2 protein were used in SPR analyses. Ligands used in the analyses were naringenin, neohesperidin, nobiletin, narirutin, rutin, and hesperidin. Biotin-streptavidin capture was performed on a commercial SA chip (Fc2) as follows for the SARS-COV-2 spike protein: 10 μg/mL in TBS-P+ buffer, 74 sec. at 5 μL/min.→˜5-5.5 kRU. Multi-cycle kinetics (MSK) analysis was performed as follows: TBS-P+ (w/0.5 μM ZnCl₂, 2% DMSO) using reference subtraction and DMSO correction. Small molecule analytes were titrated 1:2 fold over 12 concentrations from 10 μM. Association was 120 sec.@ 30 μL/min. Dissociation: 600 sec. @ 30 μL/min. An initial SPR experiment was conducted on the analytes in MCK mode from 10 μM. Hesperidin displayed binding activity with slow dissociation that confounded kinetic fitting. A follow-up SPR experiment in SCK mode from 100 UM was conducted, including halofuginone. Only the combination of rutin and hesperidin displayed binding activity, showing the synergistic effects of these two flavonoids together (FIGS. 13A-15B).

### Cytotoxicity Experiments

Calu-3 pre-treated with either inhibitor was challenged with SARS-COV-2 S1/S2 protein as follows. Calu-3 (Passage #22) cells were seeded at a density of 10,000 cells/well in clear 96-well plates in DMEM (1.5% FBS) and allowed to adhere overnight. Either hesperidin, rutin, or 1:1 ratio of hesperidin:rutin were diluted in DMEM (1.5% FBS) to 260 μM (2.6×) in 50 μL dosing volume. DMEM (1.5% FBS) with 0.5% DMSO acted as a vehicle control. Cells were incubated with inhibitors or vehicle (n=5) for 24 hours at 37° C., 5% CO₂ atmosphere. Confluency images at 10× were taken prior to dosing. Following 24-hour inhibitor pretreatment, either recombinant SARS-COV-2 spike protein (Protein S1+S2 (R683A, R685A); BioLegend, San Diego, CA, Product No. 794206) or puromycin was applied to the cells in 80 μL to the final concentration of 1 μM and 500 μM, respectively, and incubated for 48 hours. After 48-hour, incubation medium was substituted with 100 μL phenol-free IMDM media. 20 μL CellTiter 96® AQueous One Solution Cell Proliferation Assay (MTS) viability dye (Promega, Madison, WI) was added (1:6 dilution), and plates were incubated with dye for ˜3 hours at 37° C., 5% CO₂ atmosphere. Normalized viability values were generated with DMEM (+1.5% FBS) with 0.5% DMSO as 100% viable, and puromycin treated as 0% viable using GraphPad Prism 10.0.0.

Hesperidin pre-treatment with concentrations between 25 μM and 250 μM resulted in over 80% viability when normalized to SARS-COV-2 spike protein treatment alone and vehicle. However, Calu-3 viability after hesperetin+SARS-COV-2 spike protein treatment still showed a decrease of viability in a hesperetin concentration-dependent manner.

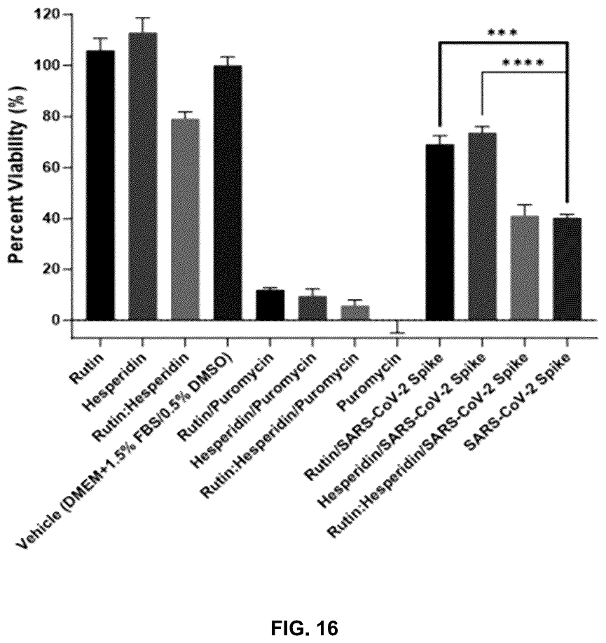

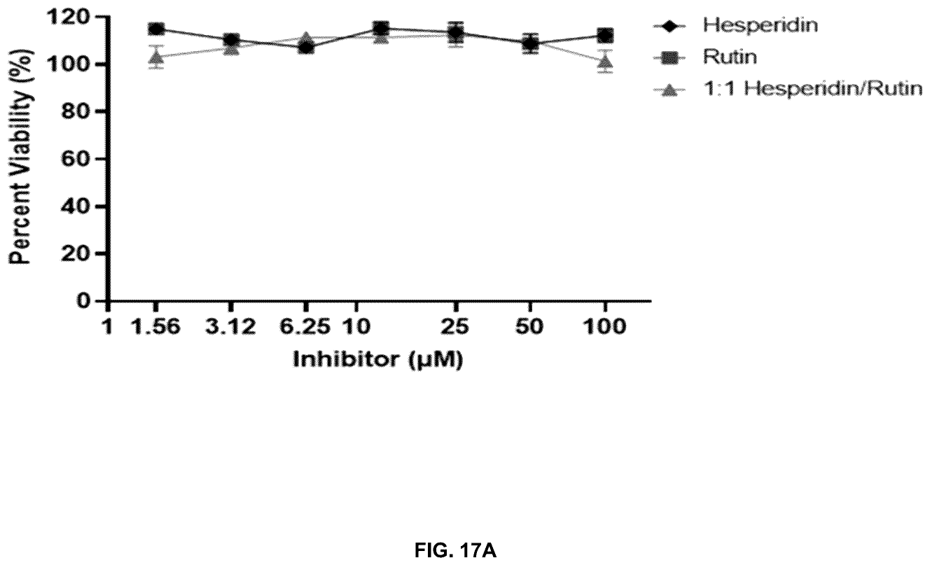

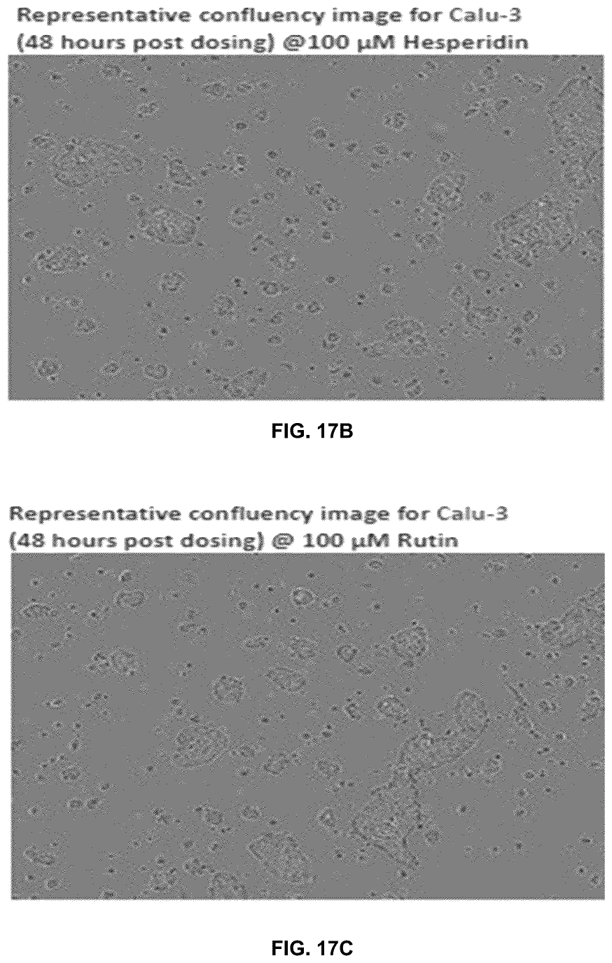

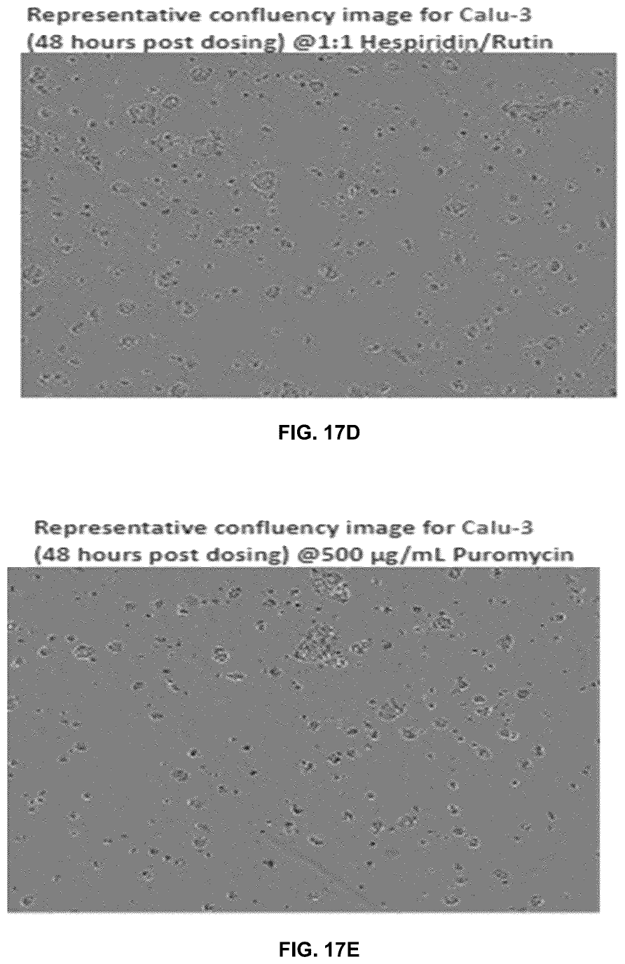

Results are Shown in FIG. 16 and FIGS. 17A-17E and Table 1 below.

TABLE 1
Normalized viability of Calu-3 cell line pre-treated
with inhibitors (100 μM) for 24 hours and incubated
with SARS-CoV-2 spike protein for 48 hours.

| | Percent viable cells (%) |
|---|---|
| | normalized to vehicle |
| Condition | control and puromycin |
| Rutin | 105.88 |
| Hesperidin | 112.93 |
| Rutin:hesperidin (1:1) | 79.20 |
| Vehicle (DMEM + 1.5% FBS/0.5% DMSO) | 100.00 |
| Rutin/puromycin | 12.02 |
| Hesperidin/puromycin | 9.57 |
| Rutin:hesperidin/puromycin | 5.80 |
| Puromycin | 0.00 |
| Rutin/SARS-CoV-2 Spike | 69.07 |
| Hesperidin/SARS-CoV-2 Spike | 73.67 |
| Rutin:hesperidin/SARS-CoV-2 Spike | 41.15 |
| SARS-CoV-2 Spike | 40.30 |

Data is normalized to 500 μg/mL puromycin and vehicle (DMEM + 1.5%) and represented as a mean and S.E.M. (N = 5). Reading is obtained from SpectraMax i3.

### CONCLUSIONS

The results show that hesperidin and rutin demonstrate binding activity against main antigen of SARS-COV 2, the spike protein. Hesperidin and rutin bind to and interfere with SARS-CoV-2 spike protein at the interface of spike-ACE2 complex, thereby blocking the main entry route viral pathogen into the pneumocytes (e.g., type 2) of the human lungs, moderating and controlling COVID infection. Flavonoids, hesperidin and rutin also bind and inactivate TMPRSS2 (PDB 7MEQ), thereby blocking the secondary route of SARS-COV 2 entry. Likewise, in the case of influenza virus, flavonoids hesperidin and rutin also bind with the viral antigens of the influenza virus, namely hemagglutinin and neuraminidase (PDB 2aeq). Hesperidin and rutin bind to sialic acid of the human upper respiratory track and interfere with propagation of influenza virus, decreasing the viral load and lessening the symptoms influenza infection. In addition, hesperidin and rutin bind to the VP1 antigen of rhinovirus and prevents it from associating with ICAM-1 and LDLR receptors to prevent and treat rhinovirus infection.

### Example 2

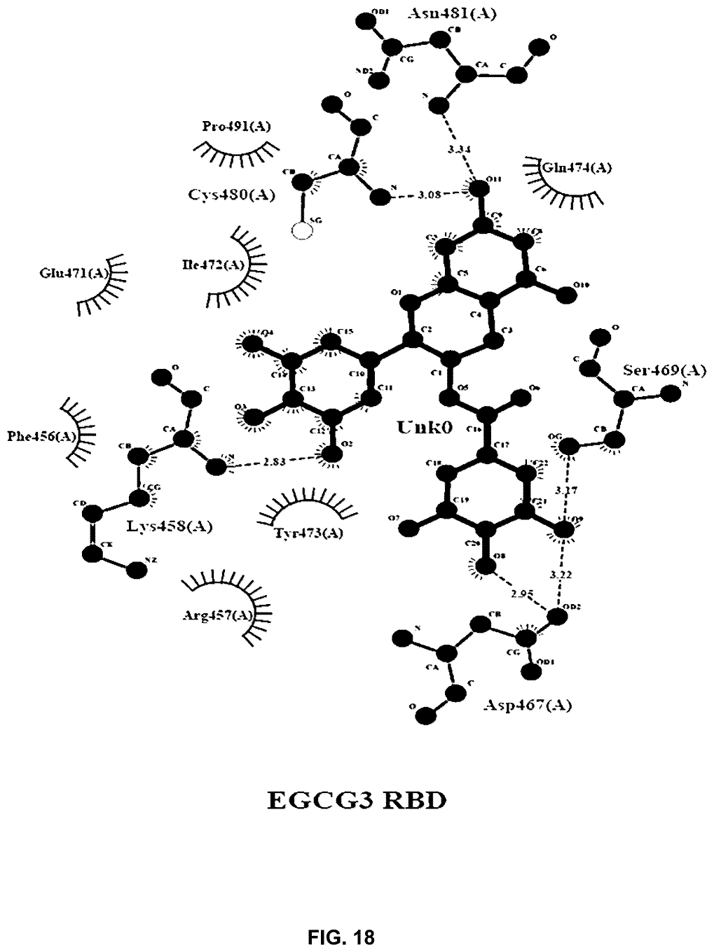

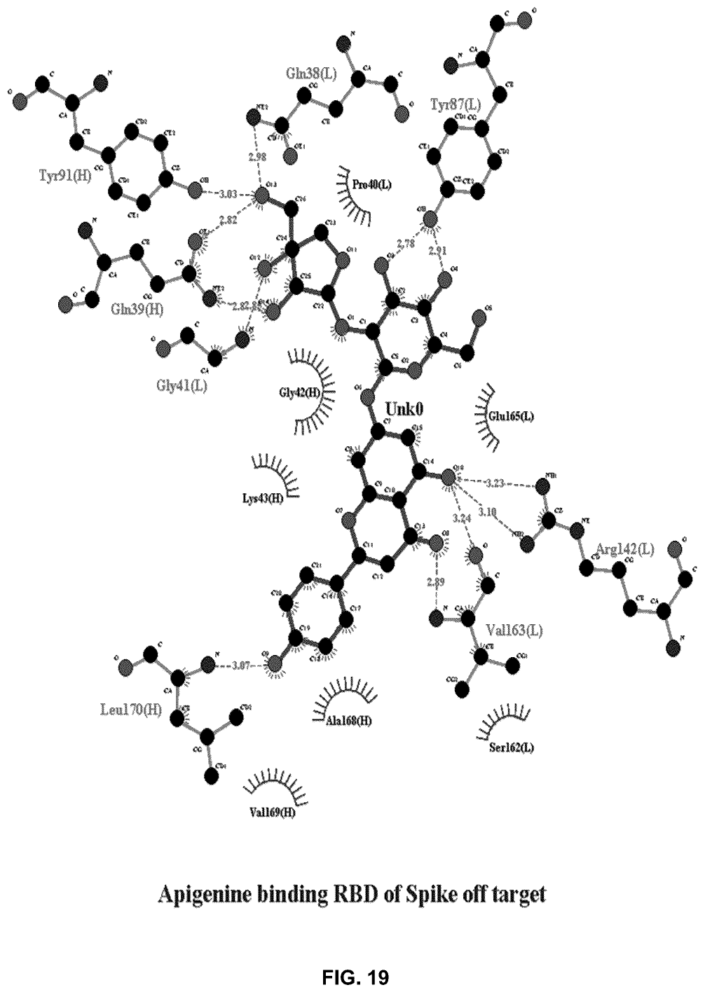

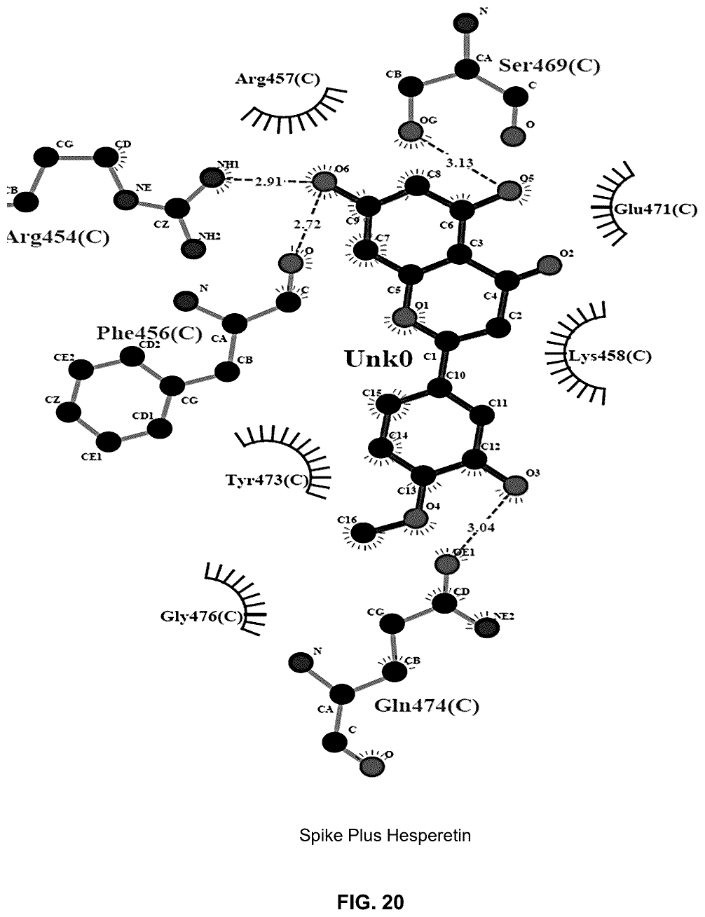

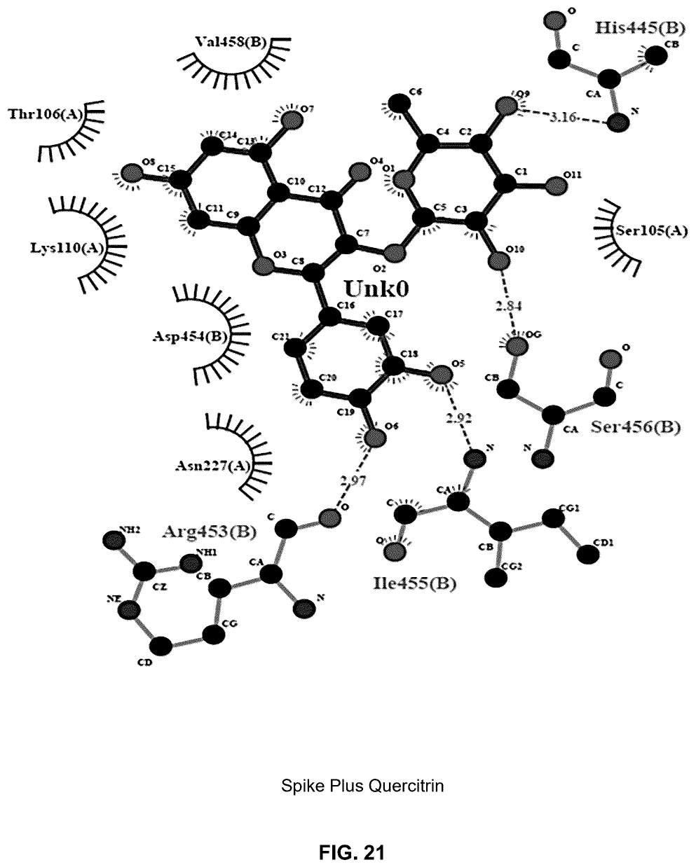

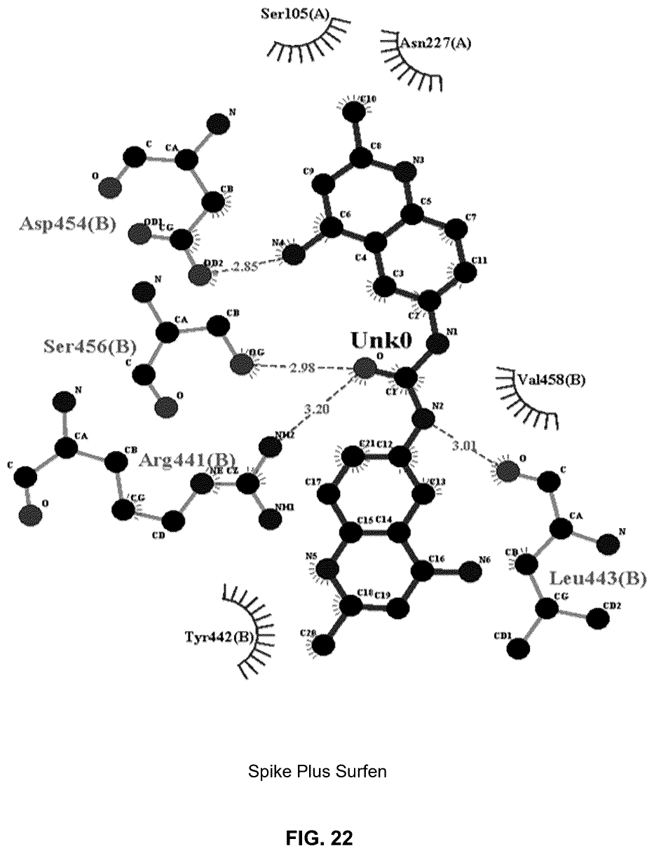

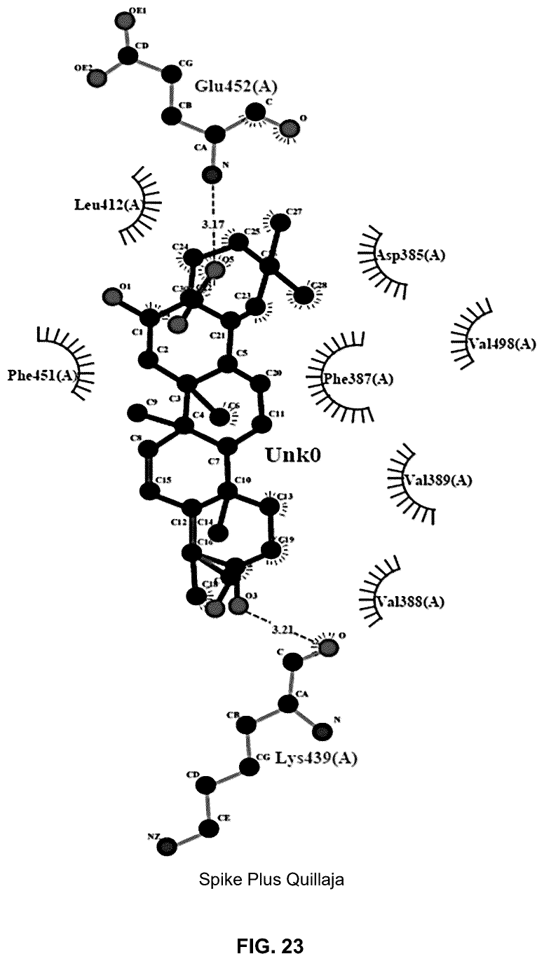

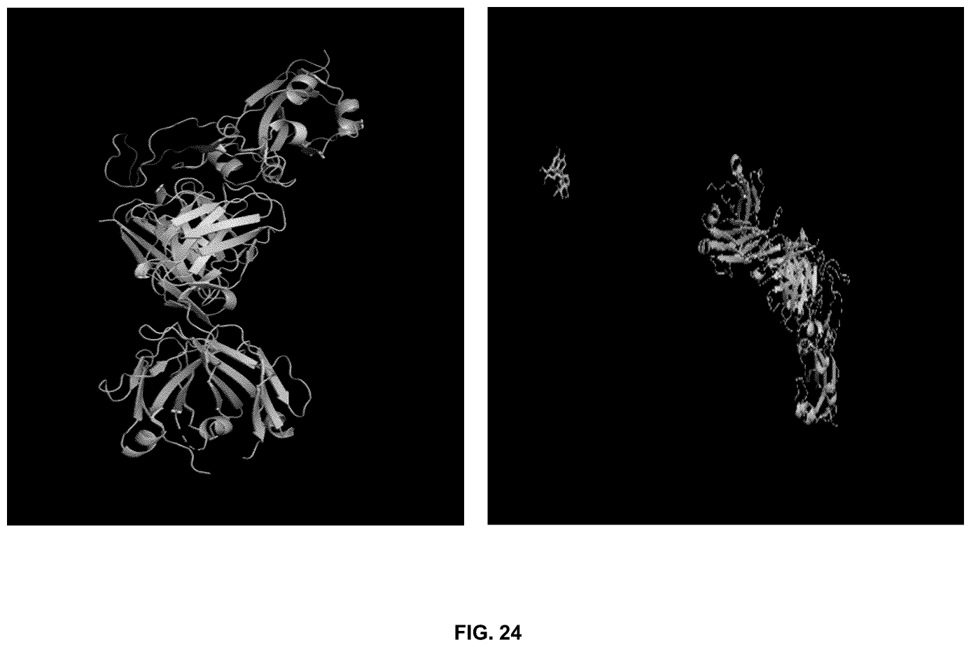

Further in silico analysis using Spike protein of SARS-COV2 with various other ligands indicates that bioactive compounds such as tangeretin (CID 68077), quercetin (CID 5280343), quercitrin (CID5280459), berberine (CID 2353), narirutin (CID 442431), and gingerol (CID 442793), epigallocatechin gallate (CID 65064) (EGCG), and surfen (CID 71166) associated strongly with the SARS-COV-2 spike protein and impedes or blocks the entry of the virus. See FIGS. 18A-24 and Table 2.

TABLE 2
In silico binding of ligands to Sars-CoV-2 spike protein.

| Species/Strains | Ligands | Affinity |
|---|---|---|
| Sars-CoV-2 Spike | Surfen | −9.2 Kcal/Mol |
| Sars-CoV-2 Spike | Quercetin | −8.7 Kcal/Mol |
| Sars-CoV-2 Spike | EGCG | −8.0 Kcal/Mol |
| Sars-CoV-2 Spike | Quercitrin | −8.5 Kcal/Mol |
| Sars-CoV-2 Spike | Apigenin | −8.4 Kcal/Mol |
| Sars-CoV-2 Spike | Quillaja | −8.7 Kcal/Mol |

AutoDock Vina docking was used for the in silico study. The AutoDock Vina docking procedure was as follows.

For the ligand preparation, the 3D structure of the ligand was obtained from PubChem or built using molecular modeling software such as Avogadro. The ligand was then energy-minimized, assigned torsions, and converted to PDBQT format using AutoDock Tools (ADT). For the protein preparation, the target protein structure was downloaded from the Protein Data Bank (PDB). Non-essential molecules such as water, ions, and alternate chains were removed. Polar hydrogen atoms were added, and the protein was converted to PDBQT format in ADT, ensuring correct atom types and charges (Gasteiger charges) were assigned.

A grid box was defined to specify the search space for the docking simulation. The center (x, y, z) coordinates and size (dimensions in Å) was set to adjust for every protein depending on their binding site.

Grid parameters are generated by Autodock Vina, which was then written in a configuration file (conf.txt), which also includes file paths for ligand and receptor. The docking was run using energy range of 4 and exhaustiveness of 8.

For the running of AutoDock Vina, docking was performed using Python language in the command Affinity (kcal/mol) explores multiple ligands poses within the defined grid.

For the output interpretation, the output file (output.pdbqt) contained docked poses ranked by binding energy. Lower (more negative) values suggested stronger binding affinity. The best pose (lowest energy) was usually selected for further analysis.

For visualization and analysis, the docked complex was visualized using software such as PyMOL, combined with LigPlot plus for evaluation.

Interaction residues were compared with known site residues to validate docking accuracy.

### Example 3

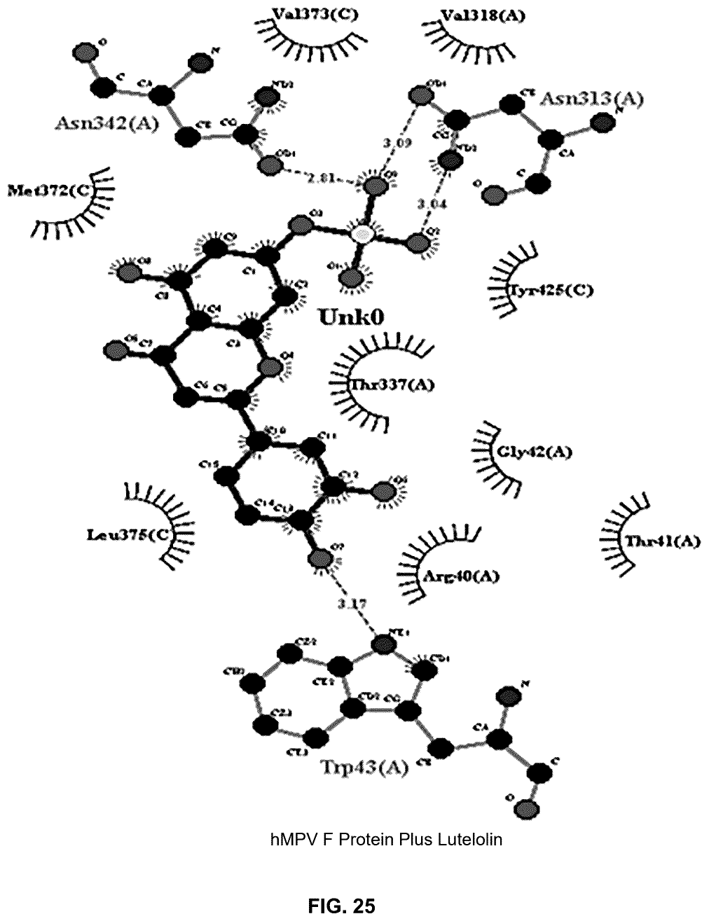

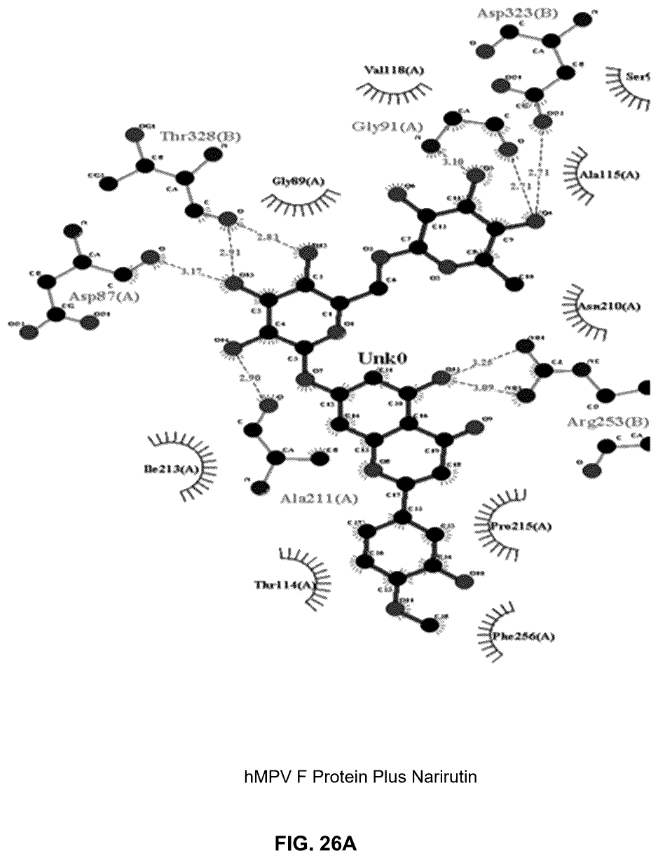

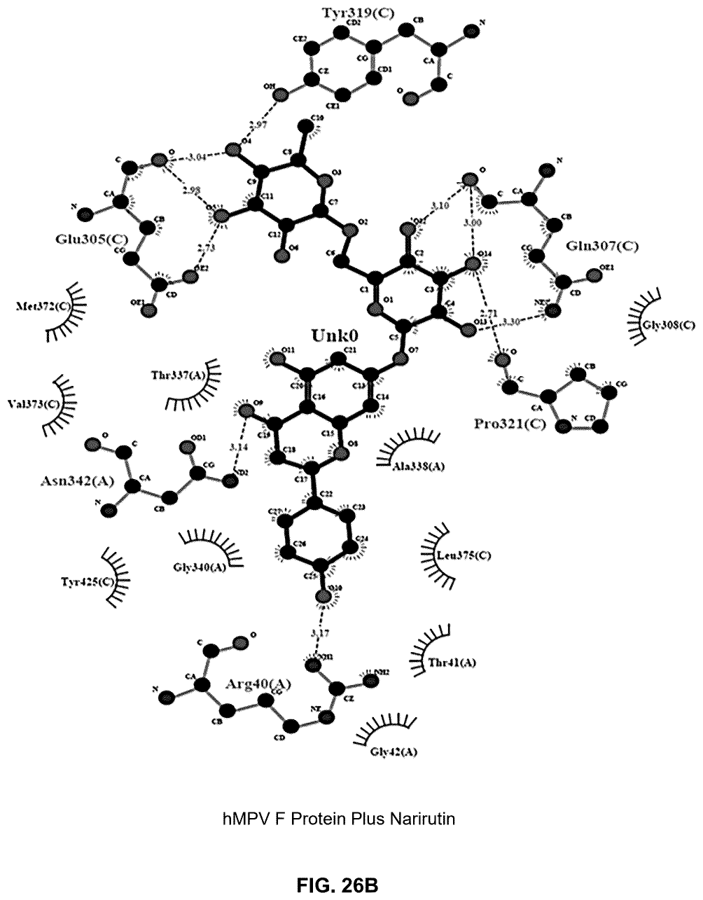

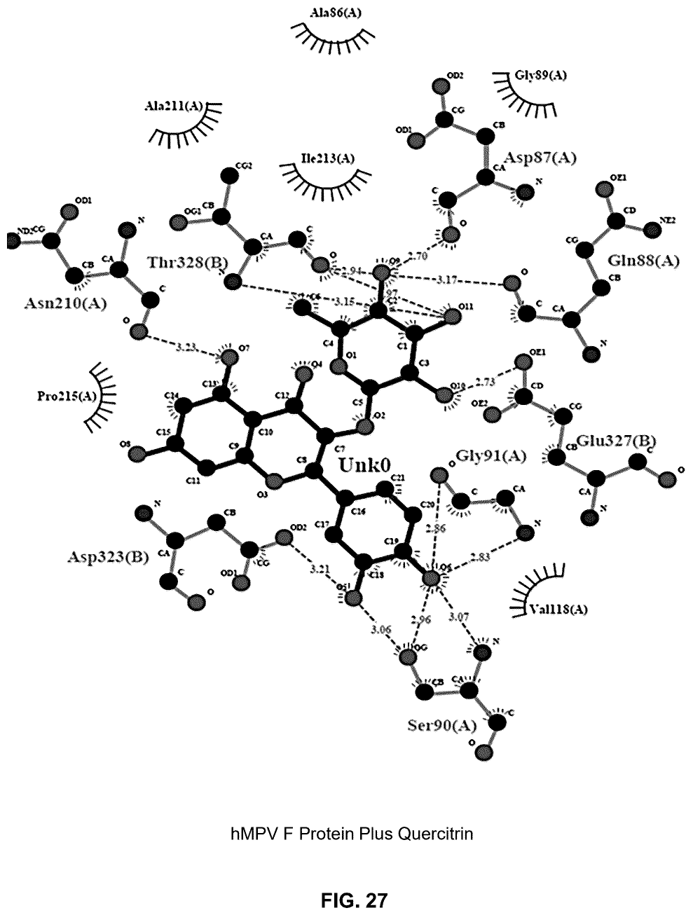

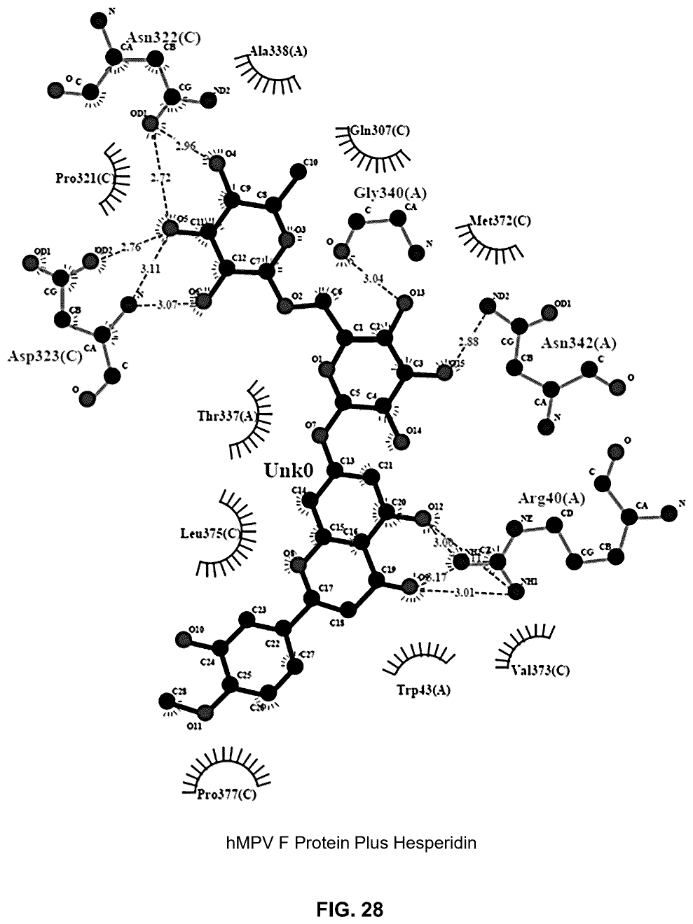

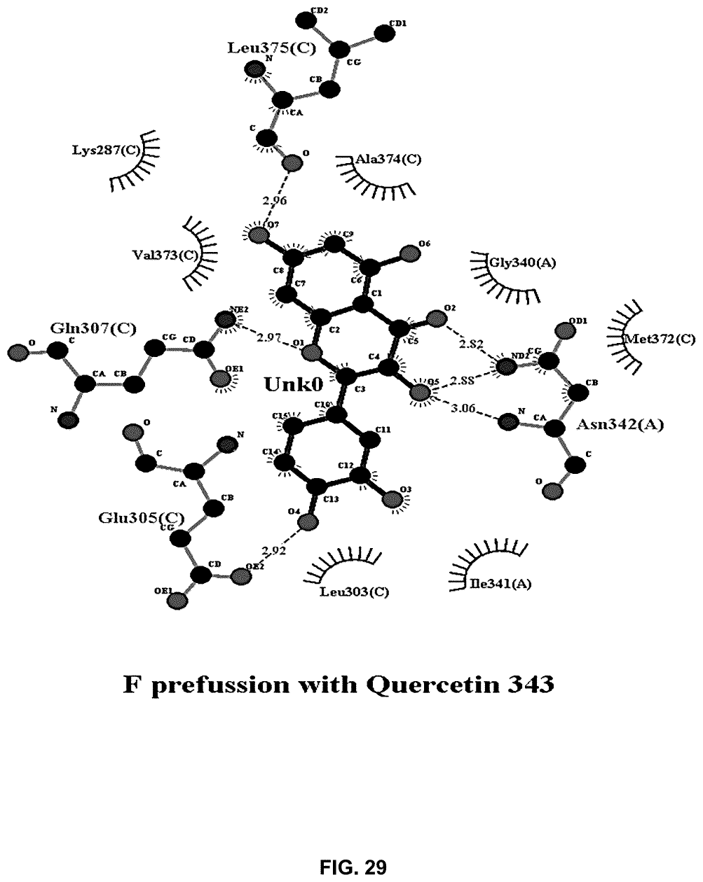

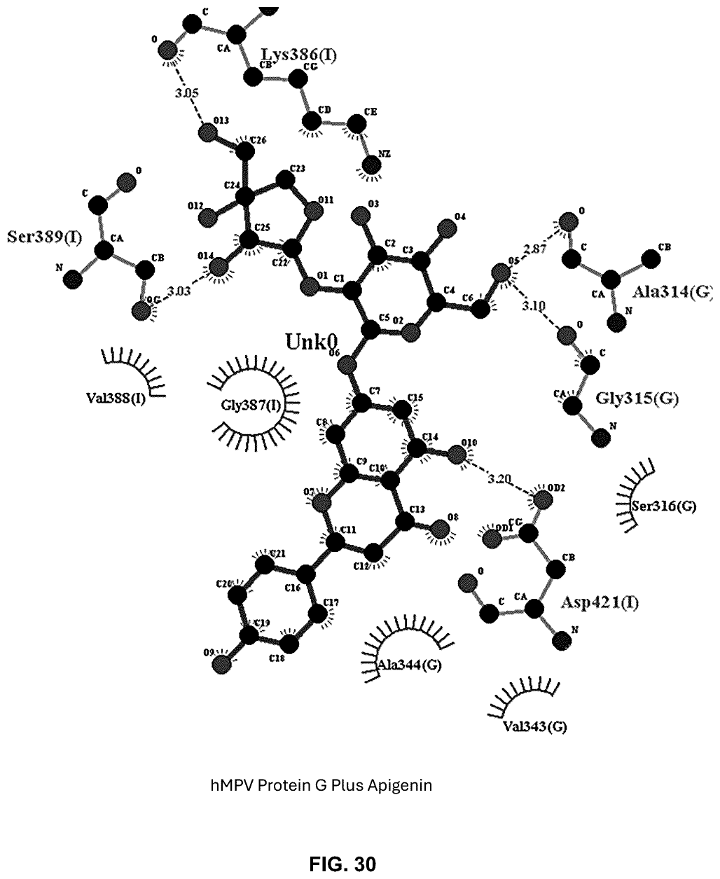

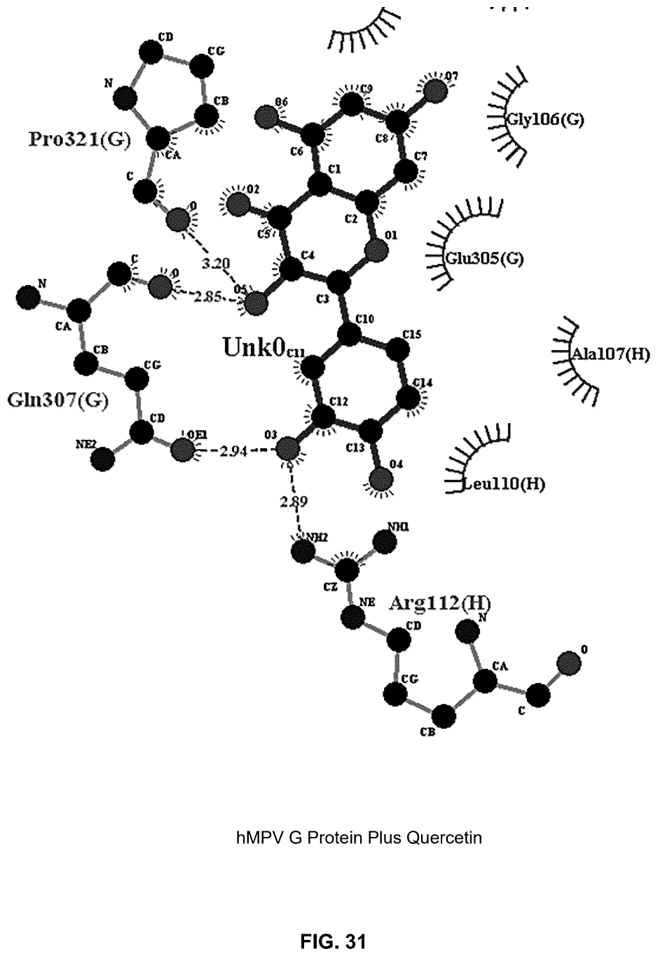

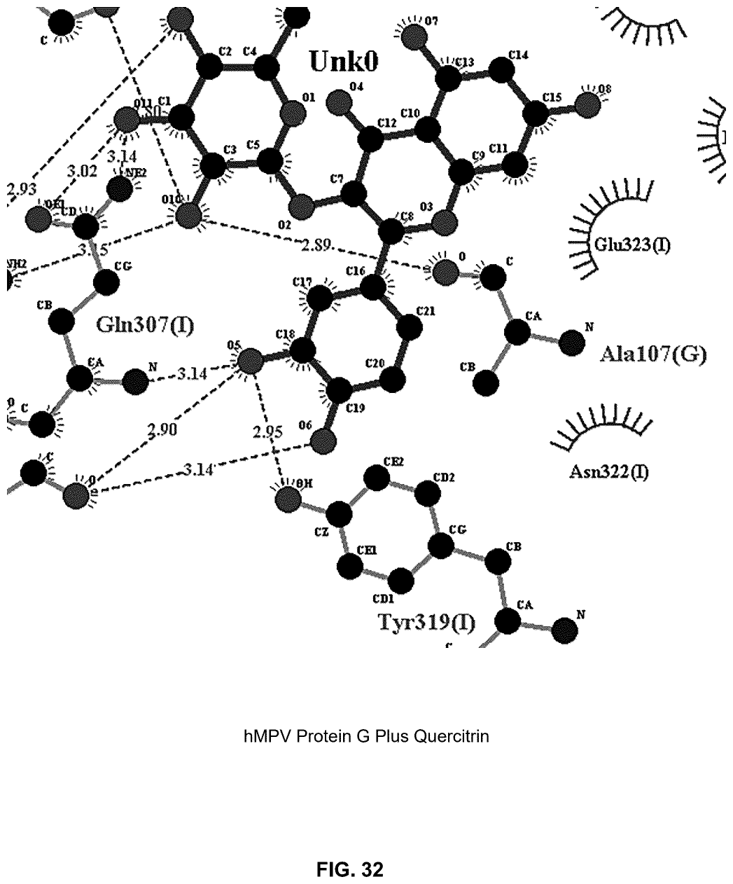

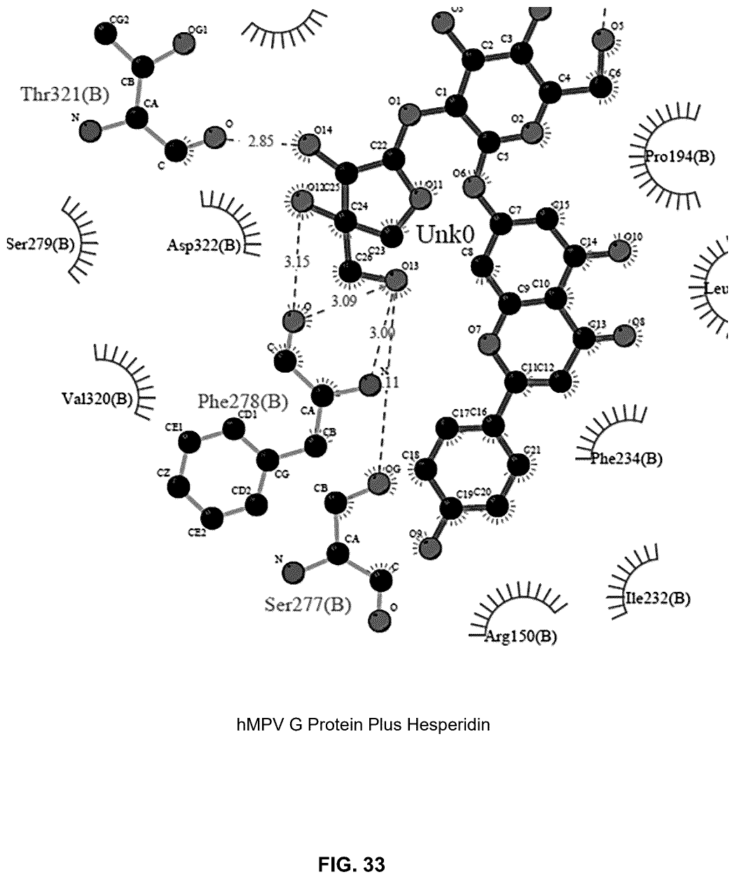

In response to reemerging of human metapneumovirus (hMPV) around the world, we investigated the effects of several bioactive compounds on the main antigens of hMPV namely fusion (F) protein. Our in silico studies indicate that apigenin (CID 5280746), resveratrol (CID 44154), hesperidin (CID 10621), narirutin (CID 4424310, quercetin (CID 580343), quercitrin (CID 580459), luteolin (CID 5280445), resveratrol, and epigallocatechin gallate (CID 65064) (EGCG) bind with F protein, while berberine, quercetin, quercitrin, and hesperidin bind with the G protein. See FIGS. 25-33 and Table 3.

TABLE 3
In silico binding of ligands to hMPV proteins.

| Species/Strains | Ligands | Affinity | |
|---|---|---|---|
| hMPV-F | Quercitrin | −8.9 | Kcal/Mol |
| hMPV-F | Narirutin | −9.5 | Kal/Mol |
| hMPV-F | Quercetin | −8.0 | Kcal/Mol |
| hMPV-F | Hesperidin | −9.4 | Kcal/Mol |
| hMPV-F | Luteolin | −7.8 | Kcal/Mol |
| hMPV-G | Hesperidin | −9.5 | Kcal/Mol |
| hMPV-G | Quercetin | −7.7 | Kcal/Mol |
| hMPV-G | Apigenin | −6.4. | Kcal/Mol |
| hMPV-G | Quercitrin | −8.6 | Kcal/Mol |
| hMPV-G | Fucoid | −5.8 | Kcal/Mol |

The data indicate that these compounds are capable of blocking the entry of Human Metapneumovirus (hMPV) and shorten the sequala of the infection by the hMPV.

AutoDock Vina docking was used for the in silico study. The AutoDock Vina docking procedure was as follows.

For the ligand preparation, the 3D structure of the ligand was obtained from PubChem or built using molecular modeling software such as Avogadro. The ligand was then energy-minimized, assigned torsions, and converted to PDBQT format using AutoDock Tools (ADT). For the protein preparation, the target protein structure was downloaded from the Protein Data Bank (PDB). Non-essential molecules such as water, ions, and alternate chains were removed. Polar hydrogen atoms were added, and the protein was converted to PDBQT format in ADT, ensuring correct atom types and charges (Gasteiger charges) were assigned.

A grid box was defined to specify the search space for the docking simulation. The center (x, y, z) coordinates and size (dimensions in Å) was set to adjust for every protein depending on their binding site.

Grid parameters are generated by Autodock Vina, which was then written in a configuration file (conf.txt), which also includes file paths for ligand and receptor. The docking was run using energy range of 4 and exhaustiveness of 8.

For the running of AutoDock Vina, docking was performed using Python language in the command Affinity (kcal/mol) explores multiple ligands poses within the defined grid.

For the output interpretation, the output file (output.pdbqt) contained docked poses ranked by binding energy. Lower (more negative) values suggested stronger binding affinity. The best pose (lowest energy) was usually selected for further analysis.

For visualization and analysis, the docked complex was visualized using software such as PyMOL, combined with LigPlot plus for evaluation.

Interaction residues were compared with known site residues to validate docking accuracy.

### Example 4

It has been discovered that many members of the dietary flavonoids contained in citrus including tangeretin, rutin, hesperidin, hesperetin, nobiletin, neohesperidin, narirutin, and naringenin prevent viral infection if taken ahead of exposure and cure viral infectivity if taken immediately after exposure. Research and laboratory testing suggests that the members of dietary flavonoids are binding with the main antigen and TMRSS2 of SARS-COV-2 and blocks the entry of coronavirus specifically, SARS-COV-2, the etiological agent of COVID. Likewise, the aforementioned flavonoids bind with the main antigen of the influenza virus, namely sialic acid, and the antigen of Rhinovirus, VP1, and blocks the common cold caused by rhinoviruses.

Two exemplary compositions containing 95% pure flavonoid compounds, namely hesperidin, hesperetin, rutin, and quercetin, plus additional agents such as piperine, N-acetylcysteine, vitamin C, vitamin D, and zinc citrate are shown in Table 4.

TABLE 4
Exemplary compositions of the invention.

| | | |
|---|---|---|
| Component | Formula 1 | Formula 2 |

| | | | | |
|---|---|---|---|---|
| Hesperidin | 250 | mg | 250-500 | mg |
| Quercetin | 250 | mg | 400-600 | mg |

| | | | |
|---|---|---|---|
| Hesperetin | 65 | mg | None |

| | | | |
|---|---|---|---|
| Rutin | None | 250-500 | mg |

| | | | | |
|---|---|---|---|---|
| Piperine (BIOPERINE ®) | 5 | mg | 5-10 | mg |
| Zinc citrate | 15 | mg | 15 | mg |
| Vitamin C (ascorbic acid) | 30 | mg | 30 | mg |
| Vitamin D3 (cholecalciferol) | 200 | IU | 200 | IU |
| N-acetylcysteine | 300 | mg | 200-400 | mg |

| | | | |
|---|---|---|---|
| Omega-3 fatty acids (EPA and DHA) | None | 250-500 | mg |

Exemplary forms of the compositions include pills, capsules, or syrup. Capsules and pills are taken orally up to 3/day, while syrup may be taken 30 ml twice a day. In some versions, Formula 1 is in the form of a capsule. In some versions, Formula 2 is in the form of a gel cap.

What is claimed is:

1. A composition comprising active agents and a carrier, wherein the active agents comprise a first set of flavonoids, wherein the first set of flavonoids consist of: hesperidin; quercetin; and hesperetin, rutin, or a combination of hesperetin and rutin.

2. The composition of claim 1, wherein the composition does not contain at least one of naringin, naringenin, diosmin, quercetin, nobiletin, and tangeretin in an amount more than 1% w/w of the combined mass of the first set of flavonoids.

3. The composition of claim 1, wherein the composition does not contain any one of naringin, naringenin, diosmin, quercetin, nobiletin, and tangeretin in an amount more than 1% w/w of the combined mass of the first set of flavonoids.

4. The composition of claim 1, wherein the composition does not contain any flavonoid other than the flavonoids in the first set of flavonoids in an amount more than 1% w/w of the combined mass of the first set of flavonoids.

5. The composition of claim 1, wherein the first set of flavonoids consists of: hesperidin; quercetin; and rutin or a combination of rutin and hesperetin, and wherein the hesperidin and the rutin are provided in the composition in amounts effective to synergize binding of at least one of the hesperidin and the rutin to SARS-COV-2 spike protein.

6. The composition of claim 1, wherein the first set of flavonoids consists of hesperidin, quercetin, and rutin.

7. The composition of claim 1, wherein the first set of flavonoids consists of hesperidin, quercetin, and hesperetin.

8. The composition of claim 1, wherein the active ingredients further comprise one or more of piperine, zinc, vitamin C, vitamin D3, N-acetylcysteine, and an omega-3 fatty acid.

9. The composition of claim 1, wherein the active ingredients consist of: hesperidin; quercetin; hesperetin, rutin, or a combination of hesperetin and rutin; piperine; a zinc salt; vitamin C; vitamin D3; N-acetylcysteine; and, optionally, one or more omega-3 fatty acids.

10. The composition of claim 1, wherein the active ingredients comprise:
hesperidin in an amount from 150 mg to 600 mg;
quercetin in an amount from 150 mg to 700 mg;
hesperetin in an amount from 1 mg to 120 mg, rutin in an amount from 150 mg to 600 mg, or a combination of hesperetin in an amount from 1 mg to 120 mg and rutin in an amount from 150 mg to 600 mg;
piperine in an amount from 1 mg to 50 mg;
a zinc salt in an amount from 1 mg to 25 mg;
vitamin C in an amount from 1 mg to 50 mg;
vitamin D3 in an amount from 60 IU to 650 IU;
N-acetylcysteine in an amount from 100 mg to 500 mg; and
optionally, one or more omega-3 fatty acids in a total amount from 150 mg to 600 mg.

11. The composition of claim 1, wherein the active ingredients consist of:
hesperidin in an amount from 150 mg to 600 mg;
quercetin in an amount from 150 mg to 700 mg;
hesperetin in an amount from 1 mg to 120 mg, rutin in an amount from 150 mg to 600 mg, or a combination of hesperetin in an amount from 1 mg to 120 mg and rutin in an amount from 150 mg to 600 mg;
piperine in an amount from 1 mg to 50 mg;
a zinc salt in an amount from 1 mg to 25 mg;
vitamin C in an amount from 1 mg to 50 mg;
vitamin D3 in an amount from 60 IU to 650 IU;
N-acetylcysteine in an amount from 100 mg to 500 mg; and
optionally, one or more omega-3 fatty acids in a total amount from 150 mg to 600 mg.

12. The composition of claim 1, wherein the composition is in a form of a capsule or a gel cap.

13. A method of treating a viral infection in a subject, the method comprising administering the composition of claim 1 to the subject in an amount effective to treat the viral infection.

14. The method of claim 13, wherein the infection is selected from the group consisting of a coronavirus infection, an influenza virus infection, a rhinovirus infection, and a human metapneumovirus infection.
